(19) United States
(12) Patent Application Publication
(10) Pub. No.: US 2019/0029970 A1
(43) Pub. Date: Jan. 31, 2019

(54) FATTY ACID CONJUGATED NANOPARTICLES AND USES THEREOF

(71) Applicant: The Chinese University of Hong Kong, Shatin (CN)

(72) Inventors: Wai Yip LEE, Ma On Shan (CN); Ho Yin Li, TaiKooShing (CN)

(21) Appl. No.: 16/046,569

(22) Filed: Jul. 26, 2018

Related U.S. Application Data
(60) Provisional application No. 62/539,255, filed on Jul. 31, 2017.

(51) Int. Cl. A61K 9/51; A61K 31/12

(52) U.S. Cl. CPC A61K 9/5123; A61K 9/5153; A61K 31/12

(57) ABSTRACT

The present invention provides fatty acid-conjugated nanoparticles and methods of making and using the same. Methods for improving delivery of therapeutic agents (e.g., drugs) contained within the fatty acid conjugated nanoparticles to the central nervous system (e.g., across the blood brain barrier) are disclosed.

### CROSS-REFERENCES TO RELATED APPLICATIONS

This application claims the benefit of U.S. Provisional Application No. 62/539,255, filed Jul. 31, 2017, the entirety of which is incorporated herein for all purposes.

### FIELD OF THE INVENTION

The disclosure generally relates to fatty acid conjugated nanoparticles containing a therapeutic agent, and delivery of the therapeutic fatty acid conjugated nanoparticles across the blood brain barrier for the treatment of disease. In some aspects, the fatty acid conjugated nanoparticles comprises a polymeric material and one or more therapeutic compounds. In some aspects, the fatty acid conjugated nanoparticles of the disclosure are particularly useful for the treatment of central nervous systems diseases and disorders of the brain such as Alzheimer's disease, Parkinson's disease, brain tumors, bacterial or viral infections and/or inflammation.

### BACKGROUND OF THE INVENTION

Diseases of the central nervous system such as Alzheimer's disease, Parkinson's disease, Huntingdon's disease, brain tumors, stroke, etc., are a growing threat due to a rapidly growing aging population and a higher life expectancy [1]. The blood-brain barrier (BBB) presents an enormous challenge in drug delivery for the treatment of these diseases. The presence of tight junctions and various efflux transporters in the BBB severely limits the entry of therapeutic agents to the brain from the systemic circulation.

Utilizing endogenous transporting systems is an attractive pathway to improve drug delivery to the brain. It is generally believed that several groups of fatty acid transporters, such as fatty acid transport protein (FATP)-1, FATP-4, and fatty acid translocase/CD36, which are expressed in human brain microvessel endothelial cells, facilitate the entry of fatty acids into the brain but the cellular uptake mechanisms remain poorly understood [2]. However, fatty acids would be attractive brain-targeting ligands due to their safety (i.e,. by the United States Food and Drug Administration) and low cost.

In addition to the BBB, poor drug solubility remains a major stumbling block affecting drug delivery to the brain. Recent advances in nanotechnology have made possible the development of novel systems for overcoming various drug delivery challenges. Of particular interest is polymeric nanoparticles that undergo self-assembly to form micellar nanoparticles.

As set forth herein, conjugation of fatty acids to polymeric nanoparticles (NPs) can potentially overcome the two major challenges in drug delivery to the brain: poor drug solubility and inefficient delivery across the BBB. Here, we report the synthesis, preparation, characterization and application of fatty acid-conjugated polymeric nanoparticles (FA-NPs) for improved drug delivery to the brain. We synthesized a library of FA-NPs by using a convergent synthetic method.

The present study employed curcumin and coumarin-6 as model compounds to demonstrate the feasibility of improving drug delivery to the brain by FA-NPs. Curcumin is derived from the rhizome of Curcuma Longa, and it is potentially used to treat neuro-degenerative and neuro-inflammatory diseases, and brain tumors [7]. Curcumin suffers from poor solubility and in vivo instability, which was attenuated using the FA-NPs described herein.

### BRIEF SUMMARY OF THE INVENTION

This invention provides new methods and compositions useful for administering a therapeutic agent (e.g., drugs and/or diagnostic agents) across the blood brain barrier, which can result in improved delivery of the therapeutic agent to a site of interest due to its encapsulation within a fatty acid conjugated nanoparticle. In one aspect, the present invention provides a novel nanoparticle with a fatty acid conjugated to the surface of the nanoparticle and containing within the nanoparticle a therapeutic agent. In some embodiments, the nanoparticle targets delivery of the therapeutic agent across the blood brain barrier into the brain.

In some embodiments, the fatty acid conjugated to the surface of the nanoparticle comprises a saturated, unsaturated, monounsaturated or polyunsaturated fatty acid. In some embodiments, the fatty acid conjugated to the surface of the nanoparticle comprises an omega-3, omega-6, or omega-9 fatty acid. In some embodiments, the omega-3 fatty acid is hexadecatrienoic acid, α-Linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid. In some embodiments, the omega-6 fatty acid is linoleic acid, gamma-linolenic acid, calendic acid, eicosadienoic acid, dihomoamma-linolenic acid, arachidonic acid, docosadienoic acid, adrenic acid, osbond acid, tetracosatetraenoic acid, and tetracosapentaenoic acid. In some embodiments, the omega-9 fatty acid is oleic acid, elaidic acid, gondoic acid, mead acid, erucic acid, nervonic acid, and ximenic acid. In some embodiments, the omega-3 fatty acid is hexadecatrienoic acid, a-Linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid. In some embodiments, the omega-6 fatty acid is linoleic acid, gamma-linolenic acid, calendic acid, eicosadienoic acid, dihomoamma-linolenic acid, arachidonic acid, docosadienoic acid, adrenic acid, osbond acid, tetracosatetraenoic acid, and tetracosapentaenoic acid. In some embodiments, the omega-9 fatty acid is oleic acid, elaidic acid, gondoic acid, mead acid, erucic acid, nervonic acid, and ximenic acid. In some embodiments, the fatty acid conjugated to the surface of the nanoparticle comprises a branched or unchained aliphatic chain. In some embodiments, the fatty acid conjugated to the surface of the nanoparticle comprises lauric acid (C12), myristic acid (C14), palmitic acid (C16), steric acid (C18), alpha-linolenic acid (ALA), linoleic acid (LA), oleic acid (OA), docosahexaenoic acid (DHA), erucic acid (EA), formic acid, acetic acid, propionic acid, butyric acid, isobutyric acid, valeric acid, isovaleric acid, and their derivatives that contain one long alkyl chain in which the number of carbon varies from 2 to 5, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, stearidonic acid, arachidonic acid and their derivatives that contain one long alkyl chain in which the number of carbon varies from 6 to 12, oxalic acid, malonic acid, succinic acid, glutaric acid, adipic acid, pimelic acid, suberic acid, azelaic acid, sebacic acid and their derivatives that contain one long alkyl chain in which the number of carbon varies from 8 to 20, citric acid, and tricarboxylic acid and its derivatives. In some embodiments, the fatty acid conjugated to the surface of the nanoparticle contains one long alkyl chain in which the number of carbons varies from 1 to 30.

In one aspect, the nanoparticle comprises a polymeric nanoparticle. In some embodiments, the polymeric nanoparticle comprises a di-block copolymer, wherein said di-block copolymer comprises (i) a first block of hydrophobic polymer and (ii) a second block of hydrophilic polymer. In some embodiments, the polymeric nanoparticle comprises a poly(ethylene glycol)-block nanoparticle. In another embodiment, the polymeric nanoparticle comprises a poly(epsilon-caprolactone) nanoparticle. In some embodiments, the polymeric nanoparticle comprises a poly(ethylene glycol)-block-poly(epsilon-caprolactone) (PEG-b-PCL) nanoparticle. In some embodiments, the length of the first block of hydrophobic polymer is between 1 and 500 repeating units (e.g., between 1 and 100, between 1 and 200, between 1 and 300, between 1 and 400, and between 1 and 450). In another embodiment, the length of the first block of the hydrophobic polymer is between 1 and 1,500 repeating units (e.g., between 1 and 100, between 1 and 200, between 1 and 300, between 1 and 400, between 1 and 500, between 1 and 600, between 1 and 700, between 1 and 800, between 1 and 900, between 1 and 1000, between 1 and 1100, between 1 and 1200, between 1 and 1300, between 1 and 1400, and between 1 and 1450). In some embodiments, the length of the second block of hydrophilic polymer is between 1 and 500 repeating units (e.g., between 1 and 100, between 1 and 200, between 1 and 300, between 1 and 400, and between 1 and 450). In another embodiment, the length of the second block of the hydrophilic polymer is between 1 and 1,500 repeating units (e.g., between 1 and 100, between 1 and 200, between 1 and 300, between 1 and 400, between 1 and 500, between 1 and 600, between 1 and 700, between 1 and 800, between 1 and 900, between 1 and 1000, between 1 and 1100, between 1 and 1200, between 1 and 1300, between 1 and 1400, and between 1 and 1450).

In another aspect, the nanoparticle comprises a plurality of polymer blocks. In some embodiments, the polymer blocks include one or more polymers comprising polylactic acid (PLA), polyethylene glycol (PEG) or polyethylene oxide (PEO), polycaprolactone (PCL), methoxypolyethylene glycol (MPEG), Poly D, L-glycolide (PLG), polycyanoacrylate (PCA), polylactic-co-glycolic acid (PLGA), polyvinyl alcohol (PVA), polyvinylpyrrolidone, polybutadiene (PBD), d-a-tocopheryl polyethylene glycol 1000 succinate, PEG-PLA, PEG-PLLLA, PEG-PDLLA, PEG-PDDLA, mPEG-PLA, mPEG-PLLLA, mPEG-PDLLA, mPEG-PDDLA, PEG-PCL, PEG-PLGA, PEG-PCL, mPEG-PCL, PEG-DPSE, mPEG-DPSE, PEO-PBD, mPEO-PBD, Pluronics (PEO-PPO-PEO), PLGA-PEG-PLGA, PEG-PLGA-PEG, PEG-PCL-PEG, PCL-PEG-PCL, Vitamin E-TPGS, Solutol HS15, and Soluplus, or a combination thereof. In some embodiments, the polymeric nanoparticle comprises a plurality of polymer blocks, wherein one or more of the polymer blocks is between 1 and 500 repeating units in length (e.g., between 1 and 100, between 1 and 200, between 1 and 300, between 1 and 400, and between 1 and 500). In another embodiment, the one or more polymer blocks is between 1 and 1,500 repeating units in length (e.g., between 1 and 100, between 1 and 200, between 1 and 300, between 1 and 400, between 1 and 500, between 1 and 600, between 1 and 700, between 1 and 800, between 1 and 900, between 1 and 1000, between 1 and 1100, between 1 and 1200, between 1 and 1300, between 1 and 1400, and between 1 and 1450). In some embodiments, the one or more polymer blocks is between 1 and 100 repeating units in length (e.g., between 1 and 10, between 1 and 20, between 1 and 30, between 1 and 40, between 1 and 50, between 1 and 60, between 1 and 70, between 1 and 80, between 1 and 90, between 1 and 95). In another embodiment, the length of the polymer blocks is different between each type of polymer (e.g., a nanoparticle having a first PEG block of 500 repeating units, and a second PCL block of 1,500 repeating units).

In another aspect, the nanoparticle comprises a liposome, solid lipid nanoparticle, gold nanoparticle, silver nanoparticle, iron nanoparticle, Gd nanoparticle, polystyrene nanoparticle, albumin nanoparticle, chitosan and derivative nanoparticles, a dendrimer, and the like.

In some embodiments, the nanoparticle has an average mean particle size of between 10 nm and 1000 nm. In some embodiments, the nanoparticle has an average mean particle size of less than 200 nm (e.g., about 5 nm to about 190 nm, about 5 nm to about 175 nm, about 5 nm to about 150 nm, about 5 nm to about 100 nm, about 5 nm to about 75 nm, about 5 nm to about 50 nm, about 10 nm to about 25 nm, about 10 nm to about 50 nm, about 10 nm to about 100 nm, about 10 nm to about 125 nm, about 10 nm to about 150 nm, about 10 nm to about 175 nm, about 25 nm to about 125 nm, about 30 nm to about 100 nm, and about 40 nm to about 80 nm). In a preferred embodiment, the nanoparticle has an average mean particle size of about 25 nm to about 125 nm. In one embodiment, the nanoparticle has an average mean particle size of about 40 nm to about 80 nm. In another embodiment, the nanoparticle has an average mean particle size of about 40 nm to about 50 nm. In some embodiments, the nanoparticle is an ultrafine polymeric nanoparticle.

In one aspect, the nanoparticle contains a therapeutic agent. In some embodiments, the therapeutic agent comprises a chemotherapeutic agent, antibiotic, antiviral drug, vaccine, diagnostic agent, monoclonal antibody or a binding fragment thereof, neuropeptide, central nervous system (CNS) stimulant, anticonvulsant, antiemetic/anti-vertigo agent, muscle relaxant, narcotic analgesic, non-narcotic analgesic, sedative, anti-inflammatory agent, cholinergic agonist, cholinesterase inhibitor, general anesthetic, and imaging agent. In some embodiments, the nanoparticle targets delivery of the therapeutic agent across the blood brain barrier.

In some embodiments, the chemotherapeutic agent contained within the nanoparticle comprises aldesleukin (proleukin), altretamine (hexalen), amsacrine, Ara-c cytarabine: cytarabine (Ara-C), anastrazole, asparaginase, azacytidine, azidothymidine, carmustine, bendamustine, bevacizumab, bromocriptine, buserelin, busulfan, cabergolin, calcium folinate (leucovorin), camptosar (irinotecan), camptosar (irinotecan), capecitabine (xeloda), carboplatin (paraplatin), CCNU (lomustine), chloramucil (leukeran), cisplatin, cladribine (leustatin), clofarabine, cytosine arabinoside, cytarabine, cytoxin (cyclophosphamide), dacarbazine, dactinomycin, daunorubicin, decitibine, dexrazoxan, docetaxel (taxotere), doxorubicin hydrochloride (hydroxydaunorubicin), epirubicin, erlotinib (tarceva), estramustine, etoposide, exemestane (aromasin), fludarabine, fluorodeoxyuridine, 5-fluorouracil, flutamide, fulvestrant, gemcitabine, goserelin (zoladex), herceptin, hydroxyurea, idarubicin, ifosfamide, imatinib, interferon, ixempra (ixabepilone), lanvis thioguanine, lapatinib ditosylate (tykerb), lenalidomide (revlimid), letrozole (femara), luprone (luprolide), lomustine, lysodren, mechlorethamine hydrochloride, mitotan, megastrol, melphalan, mesna uromitexan, mercaptopurine, methotrexate, mitomycin, mitoxantrone, mitotane, navelbine vinerelobine, nelarabine, novladex, omustine, oxaliplatin, paclitaxel, panitumumab, patipilone epithilone B, pharmorubicin epirubicin, photofrin porfimer, pentostatin, procarbazine hydrochloride (natulan), trans-Retinoic acid, rituxan (rituximab), somatuline lanreotide, streptozocin, sunitinib malate (sutent), tamoxifen, temodal temozolomide (temodar), teniposide, testosterone, topotecan, thioguanine, traztuzumab, thalidomide, thiotepa, tretinoin, vinblastine, vincristine, vepesid etoposide, vinorelbine, vindesine, vorinostat, or a combination thereof.

In one aspect, the nanoparticle has a drug loading of between 1% and 99.99% (e.g., about 1% to about 99%, about 2% to about 98%, about 3% to about 95%, about 4% to about 90%, about 5% to about 80%, about 8% to about 75%, about 10% to about 60%, about 10% to about 50%, about 15% to about 50%, about 20% to about 50%). In one embodiment, the nanoparticle has an encapsulation efficiency of between 1% and 100% (e.g., about 1% to about 99%, about 2% to about 98%, about 3% to about 95%, about 4% to about 90%, about 5% to about 80%, about 8% to about 75%, about 10% to about 60%, about 10% to about 50%, about 15% to about 50%, about 20% to about 50%). In some embodiments, the nanoparticles disclosed herein are formulated such that the therapeutic agent is released from the nanoparticles under controlled release or extended release conditions. In some embodiments, the therapeutic agent is released from the nanoparticle over a period of days, weeks or months. In some embodiments, the therapeutic agent is released over an extended time period as compared to release of a non-encapsulated therapeutic agent under identical conditions.

In another aspect, the nanoparticles disclosed herein have a polydispersity index of less than 0.5 (e.g., about 0.4, 0.3, 0.2. and 0.1). In some embodiments, the polydispersity index of the nanoparticles is less than 0.3. In some embodiments, the nanoparticle is an amphiphilic nanoparticle. In some embodiments, the nanoparticle is biodegradable. In some embodiments, the nanoparticle is a non-hemolytic and non-cytotoxic nanoparticle.

In another aspect, the nanoparticles disclosed herein have a zeta potential of between ±60 mV. In one aspect, the nanoparticles disclosed herein have a zeta potential of approximately +/−30 mV. In some embodiments, the nanoparticles disclosed herein have a zeta potential of between +/−1 mV and 20 +/−mV. In some embodiments, the nanoparticles disclosed herein have a zeta potential of between +/−1 mV and 50 +/−mV. In other embodiments, the nanoparticles disclosed herein have a zeta potential of between +/−2 mV and 30 +/−mV. In another embodiment, the nanoparticles disclosed herein have a zeta potential of between +/−5 mV and 50 +/−mV. In some embodiments, the nanoparticles disclosed herein have a zeta potential of between +/−5 mV and 30 +/−mV. In one embodiment, the nanoparticles disclosed herein have a zeta potential of between +/−10 mV and 30 +/−mV. In another embodiment, the nanoparticles have a neutral charge at the exterior surface of the nanoparticles.

In another aspect, the nanoparticles disclosed herein are formulated for injection. In some embodiments, the nanoparticles are formulated for parenteral, intravenous, intramuscular, subcutaneously, intranasal, intrathecal, intraparenchymal, intracerebroventricular, peroral, or intracranial administration.

In another aspect, the present invention provides a method for delivering a therapeutic agent by administrating to a subject in need thereof the nanoparticle described above. In some embodiments, the method is used to treat a central nervous disorder. In some cases, the method is used to treat Alzheimer's disease, Parkinson's disease, Huntingdon's disease, schizophrenia, dementia, inflammation or infectious diseases of the central nervous system, epilepsy, stroke, traumatic brain injury, encephalitis, meningitis, depression, neuroblastoma, multiple sclerosis (MS), prion disease, amyotrophic lateral sclerosis (ALS), transverse myelitis, motor neuron disease, Pick's disease, Lyme disease, brain tumors, and spinal cord tumors.

In another aspect, the present invention provides a method for delivering a therapeutic agent by administrating to a subject in need thereof the nanoparticle described above to diagnose a central nervous disorder. In some cases, the method is used to diagnose Alzheimer's disease, Parkinson's disease, Huntingdon's disease, schizophrenia, epilepsy, stroke, traumatic brain injury, encephalitis, meningitis, depression, dementia, inflammation or infectious disease of the central nervous system, neuroblastoma, multiple sclerosis (MS), prion disease, amyotrophic lateral sclerosis (ALS), transverse myelitis, motor neuron disease, Pick's disease, Lyme disease, brain tumors, and spinal cord tumors.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIG. 1 is a schematic showing an exemplary method for synthesizing FA-PEG-b-PCL nanoparticles.

FIG. 2 is a graph showing the effects of polymer concentration and solvent on mean particle size and polydispersity index.

FIG. 3 shows a microscopic image of fabricated OA-NPs particles disclosed herein.

FIG. 4 is a graph showing the stability of loaded nanoparticles in serum over a 24 hour period.

FIG. 5 is a graph showing the release profile of curcumin loaded nanoparticles as compared to free curcumin.

FIG. 6 is a graph showing brain concentrations of coumarin 6, prepared in 0.1% DMSO, PEG-NP, OA-NP, C16-NP, LA-NP, ALA-NP, and DHA-NP in rats after intravenous administration.

FIG. 7 is a chart showing the hemolytic activity of a variety of nanoparticles of the present invention.

FIGS. 8A and 8B are charts showing the effect of a variety of nanoparticles prepared as disclosed herein on cell viability as determined by MTT assay. FIG. 8A represents data obtained from bEnd. 3 cells. FIG. 8B represents data obtained from SH—SYSY cells.

FIGS. 9A and 9B are charts showing the effect of a variety of nanoparticles prepared as disclosed herein on cell viability as determined by LDH assay. FIG. 9A represents data obtained from bEnd. 3 cells. FIG. 9B represents data obtained from SH—SYSY cells.

FIGS. 10A-10J are graphs showing the critical micelle concentration (CMC) of various nanoparticles prepared as disclosed herein.

FIGS. 11A and 11B are Differential Scanning calorimetrry (DSC) chromatograms of FA-PEG-b-PCL. FIG. 11A is a DSC chromatogram of FA-PEG-b-PCL from −87° C. to 200° C. FIG. 11B is a DSC chromatogram of FA-PEG-b-PCL from −87° C. to −50° C.

FIG. 12 is a graph showing the calibration curve for GPC analysis.

FIGS. 13A-13B are GPC chromatograms for various nanoparticles of the present invention. FIG. 13A is a GPC chromatogram for PEG-b-PCL. FIG. 13B is a GPC chromatogram for OA-PEG-b-PCL.

FIGS. 14A-14B are NMR spectra for various nanoparticles of the present invention. FIG. 14A is a NMR spectra for PEG-b-PCL. FIG. 14B is a NMR spectra for OA-PEG-b-PCL.

FIGS. 15A-15B are IR spectra for various nanoparticles of the present invention. FIG. 15A is an IR spectra for PEG-b-PCL. FIG. 15B is an IR spectra for OA-PEG-b-PCL.

FIG. 16 are images of cellular uptake of fatty acid-conjugated nanoparticles using various chemical inhibitors.

FIG. 17 are images Immunofluorescent staining of fatty acid-conjugated nanoparticles in a cellular uptake assay. Top section (from left to right): phase contrast, FITC (OA-NPs), TRITC (FATP-4), FITC+TRITC overlay and phase contract+FITC+TRITC overlay. Bottom section: (from left to right): phase contrast, FITC (PEG-NPs), TRITC (FATP-4), FITC+TRITC overlay and phase contract+FITC+TRITC overlay.

FIG. 18 is a graph showing plasma pharmacokinetic data for various coumarin-6 loaded FA-NPs.

FIG. 19 is a graph showing brain pharmacokinetic data for various coumarin-6 loaded FA-NPs.

FIG. 20 is a graph showing accumulation of various coumarin-6 loaded FA-NP formulations in different organs.

FIG. 21 is a graph showing accumulation of various coumarin-6 loaded FA-NPs in distinct regions of the brain.

### DEFINITIONS

As used herein, the terms “a” or “an”, when used in reference to an “agent” or a “therapeutic” agent, means at least one. For example, where a fatty acid conjugated nanoparticle comprises a therapeutic agent, the fatty acid conjugated nanoparticle contains at least one therapeutic agent. In another example, “a” therapeutic agent can comprise two or more therapeutic agents (e.g., one or more drugs and one or more diagnostic agents).

Unless otherwise stated, the term “average” is synonymous with “mean” in the specification herein and has an ordinary meaning in the art. Further, unless otherwise stated, “particle size” and “particle diameter” are synonymous in the specification herein and can be measured by methods known in the art, which include but are not limited to light-scattering methods and microscopy.

As used herein, the term “amount” as used in the context of the amount of a particular therapeutic agent, refers to the concentration, quantity, percentage, or relative amount of the therapeutic agent.

As used herein, the term “agent” refers to any molecule, compound, and/or substance for use in the prevention, treatment, management, imaging, and/or diagnosis of a disease, including but not limited to central nervous disorders, including but not limited to, traumatic brain injury, encephalitis, meningitis, Alzheimer's disease, Parkinson's disease, Huntington's disease, depression, stroke, neuroblastomas, brain tumors and spinal cord tumors. In some embodiments, the agent can include an imaging biomarker that allows imaging of specific organs, tissues, cells, diseases or physiological functions. In some embodiments, the agent is specific for brain or spinal cord tumors, or traumatic brain injuries. As used herein, the term “therapeutic agent” refers to a compound or molecule that, when present in an effective amount, produces a desired therapeutic effect in a subject in need thereof. The present invention contemplates a broad range of therapeutic agents and their use with the disclosed nanoparticles. In some embodiments, the therapeutic agent can include a chemotherapeutic agent (e.g., cisplatin), an antibiotic (e.g., amoxicillin), a vaccine (e.g., Hepatitis B vaccine), a monoclonal antibody or binding fragment thereof (e.g., crenezumab), a neuropeptide (e.g., neurokinins), or an imaging agent (e.g., coumarin 6). In another embodiment, the therapeutic agent can include an anticonvulsant, antiemetic/anti-vertigo agent, anti-Parkinson agent, anti-Huntingdon's agent, anti-Alzheimer's agent, CNS stimulant, muscle relaxant, narcotic analgesic (pain reliever), nonnarcotic analgesic (such as acetaminophen and NSAID), a sedative, cholinergic agonist, cholinesterase inhibitor, general anesthetic, addiction treatment drug (such as alcohol-dependency), and the like. In a preferred embodiment, the therapeutic agent is suitable for the treatment of a CNS disorder.

As used herein, the term “cancer” refers to a neoplasm or tumor resulting from abnormal uncontrolled growth of cells. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, cancer refers to a malignant tumor, which has invaded and destroyed neighboring body structures and spread to distant sites (i.e., metastatic). In yet other embodiments, the cancer is associated with a specific cancer antigen. In some embodiments, the cancer is associated with the brain and/or spinal cord.

As used herein, the term “cancer cells” refers to cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis. The term “cancer cell” is meant to encompass both pre-malignant and malignant cancer cells. In some embodiments, the cancer cells include a pre-malignant or malignant brain cell.

As used herein, the term “cytotoxic” or the phrase “cytotoxicity” refers to the quality of a compound to cause adverse effects on cell growth or viability. The “adverse effects” included in this definition are cell death and impairment of cells with respect to growth, longevity, or proliferative activity. In some embodiments, cytotoxicity is measured as a value of cell hemolysis.

As used herein, the terms “disorder” and “disease” are used interchangeably to refer to a pathological condition in a subject. In some embodiments, disorders suitable for treatment by the claimed compositions and methods include central nervous system (CNS) disorders.

A “disease of the CNS” or “CNS disorder” encompasses any condition that affects the brain and/or spinal cord and that leads to suboptimal function. In some embodiments, the CNS disorder is an acute disorder. Acute disorders of the CNS include focal brain ischemia, global brain ischemia, brain trauma, spinal cord injury, acute infections, status epilepticus (SE), migraine headache, acute psychosis, suicidal depression, and acute anxiety/phobia. In some embodiments, the CNS disorder is a chronic disorder. Chronic disorders of the CNS include chronic neurodegeneration, retinal degeneration, depression, chronic affective disorders, lysosmal storage disorders, chronic infections of the brain, brain cancer, stroke rehabilitation, autism, mental retardation. Chronic neurodegeneration includes neurodegenerative diseases such as prion diseases, Alzheimer's disease (AD), Parkinson's disease (PD), Huntington's disease (HD), multiple sclerosis (MS), amyotrophic lateral sclerosis (ALS), transverse myelitis, motor neuron disease, Pick's disease, tuberous sclerosis, lysosomal storage disorders, Canavan's disease, Rett's syndrome, spinocerebellar ataxias, Friedreich's ataxia, optic atrophy, retinal degeneration, and aging of the CNS.

As used herein, the term “effective amount” refers to the amount of a therapeutic agent that is sufficient to result in the prevention of the development, recurrence, or onset of a disease and one or more symptoms thereof, to enhance or improve the prophylactic effect(s) of another therapy, reduce the severity, the duration of a disease, ameliorate one or more symptoms of a disease, prevent the advancement of a disease, cause regression of a disease, and/or enhance or improve the therapeutic effect(s) of another therapy.

As used herein, the terms “treat,” “treatment,” and “treating” refer to an amelioration of a disease or disorder, or at least one discernible symptom thereof. In another embodiment, “treat,” “treatment,” or “treating” refers to an amelioration of at least one measurable physical parameter, not necessarily discernible by the patient. In yet another embodiment, “treat,” “treatment,” or “treating” refers to inhibiting the progression of a disease or disorder, either physically, e.g., stabilization of a discernible symptom, physiologically, e.g., stabilization of a physical parameter, or both. In yet another embodiment, “treat,” “treatment,” or “treating” refers to delaying the onset of a disease or disorder. In one embodiment, the terms “treat,” “treatment,” and “treating” refer to the amelioration of a CNS disorder or disease.

As used herein, the term “fatty acid” refers to a carboxylic acid with an aliphatic chain (e.g., C₁ to C₃₀), which is either saturated (i.e., absence of carbon-carbon double bonds) or unsaturated (i.e., presence of carbon-carbon double bonds). The fatty acid can include an unbranched or branched aliphatic chain. In some embodiments, a fatty acid can include an aliphatic chain of fewer than six carbons (e.g., butyric acid). In another embodiment, a fatty acid can be an aliphatic chain of 6 to 12 carbons. In yet another embodiment, a fatty acid can be an aliphatic chain of 13 to 21 carbons. In some embodiments, a fatty acid can be a C₄-C₂₁ fatty acid. In some embodiments, a fatty acid can be unsaturated. In some embodiments, a fatty acid group can be monounsaturated. In some embodiments, a fatty acid group can be polyunsaturated. In some embodiments, a double bond of an unsaturated fatty acid group can be in the cis conformation. In some embodiments, a double bond of an unsaturated fatty acid can be in the trans conformation. In one embodiment, a fatty acid can include an omega-3, omega-6, or omega-9 fatty acid. In another embodiment, a fatty acid can include one or more of butyric, caproic, caprylic, capric, lauric, myristic, palmitic, stearic, arachidic, behenic, and lignoceric acid. In yet another embodiment, a fatty acid can include one or more of palmitoleic, oleic, vaccenic, linoleic, alpha-linolenic, gamma-linoleic, arachidonic, gadoleic, arachidonic, eicosapentaenoic, docosahexaenoic, and erucic acid. In some embodiments, a fatty acid can include one or more of formic acid, acetic acid, propionic acid, butyric acid, isobutyric acid, valeric acid, and isovaleric acid. In another embodiment, a fatty acid can include an oxalic acid, malonic acid, succinic acid, glutaric acid, adipic acid, pimelic acid, suberic acid, azelaic acid, and sebacic acid.

As used herein, the term “polymer” refers to a molecular structure comprising one or more repeat units (monomers), connected by covalent bonds. The repeat units may all be identical, or in some cases, there may be more than one type of repeat unit present within the polymer. If more than one type of repeat unit is present within the polymer, then the polymer is said to be a “copolymer.” The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks. A block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In some embodiments, the polymer (e.g., diblock copolymer) can be amphiphilic, i.e., having a hydrophilic portion and a hydrophobic portion. A hydrophilic polymer can be a polymer that generally attracts water and a hydrophobic polymer can be a polymer that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about) 60°. In some cases, the hydrophilicity of two or more polymers may be measured relative to each other, i.e., a first polymer may be more hydrophilic than a second polymer.

As used herein, the term “nanoparticle” refers to a particle having an average mean particle size in the nanoscale, i.e., less than 1000 nm. In particular embodiments, the term nanoparticle includes a liposome, solid lipid nanoparticle, gold nanoparticle, silver nanoparticle, iron nanoparticle, Gd nanoparticle, polystyrene nanoparticle, albumin nanoparticle, chitosan and derivative nanoparticles, a dendrimer, and a polymeric nanoparticle.

As used herein, the term “polymeric nanoparticle” refers to a nanoparticle as defined herein that comprises or consists of one or more polymers. In one aspect, the polymeric nanoparticle forms a colloidal suspension or dispersion in aqueous solution. In some embodiments, the polymeric nanoparticle is comprised of diblock copolymers. In one embodiment, the diblock copolymer comprises a poly(ethylene glycol)-block-poly(epsilon-caprolactone) (PEG-b-PCL) nanoparticle. In some embodiments, the polymeric nanoparticle self-assembles into a micelle nanoparticle. In some aspects, the polymeric nanoparticle is amphiphilic. In some embodiments, polymeric nanoparticles of the present invention are polymerized prior to conjugation of a fatty acid to the exterior surface of the polymeric nanoparticle. In several embodiments, the polymeric nanoparticle further includes one or more therapeutic agents located, solubilized, entrapped or encapsulated within the polymeric nanoparticle.

As used herein, the term “ultrafine polymeric nanoparticles” refers to a plurality of polymeric nanoparticles as defined above, having an average mean particle size of between 1 nm and 100 nm.

As used herein, the term “conjugated,” “conjugate,” or “conjugation,” when used with respect to two or more moieties, refers to moieties that are physically associated or connected with one another, either directly or via one or more additional moieties that serves as a linking agent, to form a structure that is sufficiently stable so that the moieties remain physically associated under the conditions in which structure is used, e.g., physiological conditions. In some embodiments, the moieties are attached to one another by one or more covalent bonds. In some embodiments, the moieties are attached to one another by a mechanism that involves specific (but non-covalent) binding (e.g., streptavidin/avidin interactions). In one embodiment, conjugation includes a chemical process by which a fatty acid is covalently linked to a polymeric nanoparticle of the invention. In some embodiments, conjugation includes physically adsorbing a fatty acid on to the exterior surface of the nanoparticle. According to the methods and compositions disclosed herein, fatty acids may be attached to the nanoparticle by any means known in the art. Conjugation methods include chemical complexing, which may be either ionic or non-ionic in nature, or covalent binding. Conjugation of fatty acids to a nanoparticle may occur to reactive head groups of individual lipid monomers for example, in liposomes or a collection of lipid monomers prior to assembly of the nanoparticle. Alternatively, a fatty acid can be attached to the exterior surface of a nanoparticle after the nanoparticle is formed.

As used herein, the term “polydispersity index” or “PDI” refers to the size distribution of a population of particles. In some embodiments, PDI refers to the size distribution of a population of polymer nanoparticles. Polydispersity index can be determined by a number of techniques including dynamic light scattering (DLS), quasi-elastic light scattering (QELS), and electron microscopy. Polydispersity index (PDI) is usually calculated as:

$$PDI = {( \frac{σ}{d} )}^{2}$$

i.e., the square of (standard deviation/mean diameter).

As used herein, the term “zeta potential” refers to electrokinetic potential in colloidal dispersions. It is denoted by the Greek letter, “ζ”. Generally, zeta potential is the potential difference between the dispersion medium and the stationary layer of fluid attached to the dispersed particle. The magnitude of the zeta potential indicates the degree of electrostatic repulsion between adjacent, similarly charged particles in a dispersion. The zeta potential is frequently used as an indicator of the stability of colloidal dispersions. In some instances, a high zeta potential can suggest stability (i.e, the solution or dispersion will resist aggregation). However, when the zeta potential is small, attractive forces may exceed this repulsion and the dispersion may break and flocculate. Thus, colloids with a high zeta potential (negative or positive) are generally electrically stable while colloids with low zeta potentials tend to coagulate or flocculate. In some embodiments, the nanoparticles disclosed herein comprise stable colloids having a high zeta potential (e.g., from +/−30 mV to more than 60 mV).

As used herein, the term “drug loading” is the process of incorporating a therapeutic agent into a nanoparticle. Drug loading can be expressed as:

$$DL ( \% ) = \frac{weight of the drug in supernatant}{weight of the drug and polymer added} \times 100 \%$$

Traditional methods for detecting drug loading include UV, mass spectrometry, fluorescent detection, protein content (Bradford method), reflective index, ELISA, among other methods.

As used herein, “encapsulation efficiency” or “EE” refers to the percentage of therapeutic agent that is successfully encapsulate or localized within or on the nanoparticles. Generally, it can be calculated as follows:

$$EE ( \% ) = \frac{weight of the drug in supernatant}{weight of the drug added} \times 100 \%$$

Accordingly, if the EE is 30% this means that 30% of the original amount of therapeutic agent is encapsulated or localized within or on the nanoparticles.

As used herein, the terms “delivery” and “delivering” refer to conveyance of a therapeutic agent to a subject using the methods of the invention. Delivery may be localized to a particular location in a subject, such as a tissue, an organ, or cells of a particular type. In some embodiments, delivering includes localization of a therapeutic agent across the blood brain barrier including, but not limited to, brain cells or cerebral spinal fluid (CSF).

As used herein, the term “subject” refers to any mammal, in particular a human, at any stage of life.

As used herein, the term “consists essentially of” refers to a composition having the stated components, in addition to minor components (e.g., unavoidable impurities) that do not materially affect the properties of the composition (e.g., the average size or dispersity of a population of nanoparticles).

As used herein, the term “about” indicates a range of +/−10% around a numerical value when used to modify that specific value.

Concentrations, amounts, cell counts, percentages, and other numerical values may be presented herein in a range format. It is to be understood that such a range format is used merely for convenience and brevity and should be interpreted to include not only the numerical values explicitly recited as the limits of the range but also to include all the individual numerical values or sub-ranges encompassed within that range as if each numerical value and sub-range is explicitly recited.

### DETAILED DESCRIPTION OF THE INVENTION

### I. Introduction

The blood brain barrier (BBB) is a limiting factor in the delivery of many peripherally-administered agents to the central nervous system. The present disclosure provides a nanoparticle with a fatty acid conjugated to the surface of the nanoparticle (FA-NP) and containing within the nanoparticle a therapeutic agent, that is able to cross the BBB, and retain its activity once across the BBB. Various aspects of the invention address the limiting factors of delivery, by providing fatty acid conjugated nanoparticles (FA-NPs) that have one or more therapeutic agents associated therewith. In short, the FA-NPs disclosed herein improve the delivery of therapeutic agents to a site of interest due to their encapsulation within the fatty acid conjugated nanoparticle. The disclosed FA-NPs provide surprising stability and improved delivery of therapeutic agents that are generally considered poorly soluble or therapeutically ineffective because of low dosages at the site of interest after administration to a subject in need thereof

In general, the disclosure provides a fatty acid-conjugated nanoparticle (FA-NP), wherein a therapeutic agent is loaded onto the FA-NP. In some embodiments, the therapeutic agent is located within the nanoparticle. The nanoparticles produced by the methods disclosed herein exhibit a small particle size (typically less than 200 nm in average or mean particle size), and narrow size distribution (low polydispersity index, typically less than 0.5), making them useful in a wide range of applications as discussed herein.

A. Components and Methods for Making Fatty Acid-Nanoparticles (FA-NPs)

The present invention discloses a nanoparticle having one or more fatty acids conjugated to the surface thereof and containing within the nanoparticle a therapeutic agent. The shape, size and chemical composition of the nanoparticle contributes to the physical and physiochemical properties of the resulting fatty acid-conjugated nanoparticle. These properties include, for example, optical properties, optoelectronic properties, electrochemical properties, electronic properties, stability in various solutions, magnetic properties, polydispersity index, molecular weight, and pore and channel size variation. Any suitable nanoparticle may be used to perform the claimed methods or prepare the various fatty acid conjugated nanoparticles disclosed herein.

In one embodiment, mixtures of nanoparticles having different sizes, shapes and/or chemical compositions, as well as the use of nanoparticles having uniform sizes, shapes and chemical composition, and therefore a mixture of properties are contemplated. Examples of suitable nanoparticles include, without limitation, spherical nanoparticles, non-spherical rods, tetrahedral, and/or prisms and core-shell nanoparticles, such as those described in U.S. Pat. No. 9,161,962; US20100092761 and US20140044791.

In one embodiment, the nanoparticle is metallic, and in various aspects, the nanoparticle is a colloidal metal. Thus, in various embodiments, nanoparticles of the invention include metal (including for example and without limitation, silver, gold, iron, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

In one embodiment, the nanoparticle is non-metallic, such as a liposome (e.g., US Patent Application No. 20130028962), albumin nanoparticle (e.g., US Patent Application No. 20140186447), chitosan and derivative nanoparticles (e.g., U.S. Pat. No. 7,740,883), and dendrimers.

Nanoparticles of the invention include those that are commercially available (See for example, U.S. Patent Publication No 2003/0147966), as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4) : 1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuC14 and a citrate-reducing agent, using methods known in the art. See, e.g., Marinakos et al, Adv. Mater. 11:34-37(1999); Marinakos et al, Chem. Mater. 10: 1214-19(1998); Enustun & Turkevich, J. Am. Chem. Soc. 85: 3317(1963).

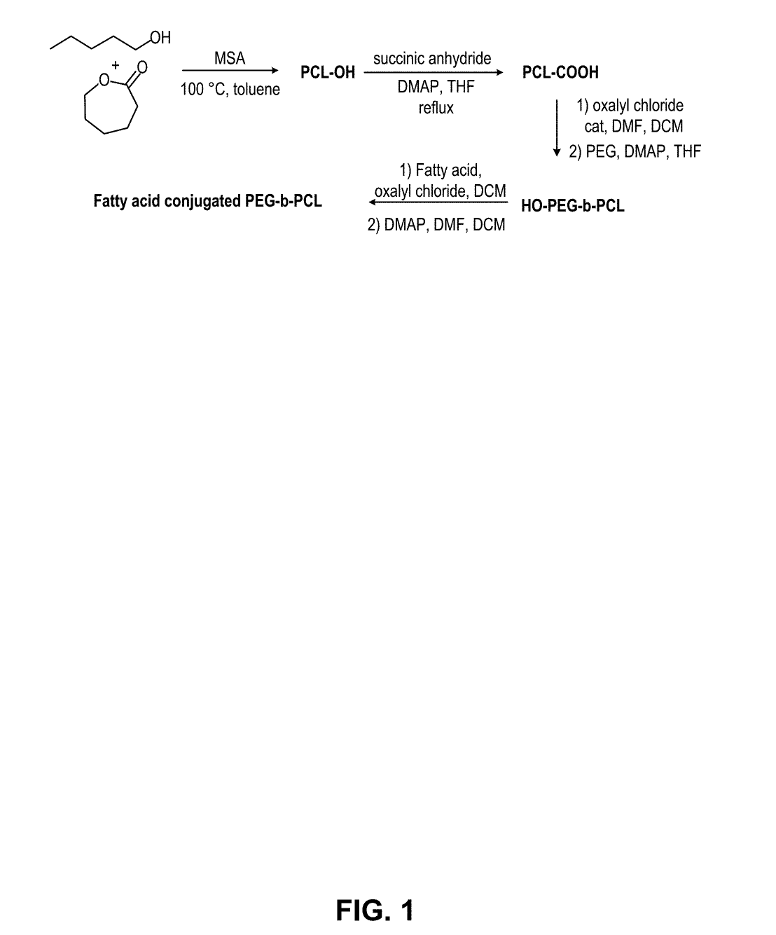

Various techniques are known for producing nanoparticles. The nanoparticles of the invention can be prepared by any suitable method, including but not limited to, the convergent synthesis method set forth in FIG. 1 and the examples. In one such embodiment, the fatty acid is conjugated to the surface of the nanoparticle after formation of a polymeric nanomaterial (e.g., PEG-b-PCL). In one exemplary embodiment, the nanoparticle comprises a mono hydroxyl-functionalized PEG-b-PCL prior to attachment of the fatty acid to the exterior surface of the nanoparticle.

In some embodiments, the nanoparticle is a polymeric nanoparticle. Various polymers are well-known in the nanoparticle field, such as PEG, Pluronics, PLGA and PLA. Any suitable polymer may be used to prepare the nanoparticles described herein. In some embodiments, the nanoparticle comprises a plurality of polymer blocks, such as a diblock copolymer. In some embodiments, a diblock copolymer comprises (i) a first block of hydrophobic polymer and (ii) a second block of hydrophilic polymer that can be used to prepare the nanoparticles disclosed herein.

In one embodiment, the polymeric nanoparticle comprises a poly(ethylene glycol)-block nanoparticle. In another embodiment, the polymeric nanoparticle comprises a poly(epsilon-caprolactone) nanoparticle. In yet another embodiment, the polymeric nanoparticle comprises a poly(ethylene glycol)-block-poly(epsilon-caprolactone) (PEG-b-PCL) nanoparticle.

In one aspect, the length of the polymer blocks can be varied according to the proposed application. In some embodiments, the polymers blocks are selected from the group consisting of polylactic acid (PLA), polyethylene glycol (PEG) or polyethylene oxide (PEO), polycaprolactone (PCL), methoxypolyethylene glycol (MPEG), Poly D, L-glycolide (PLG), polycyanoacrylate (PCA), polylactic-co-glycolic acid (PLGA), polyvinyl alcohol (PVA), polyvinylpyrrolidone, polybutadiene (PBD), methyl methacrylate(MMA), methacrylic acid (MAA), d-α-tocopheryl polyethylene glycol 1000 succinate, PEG-PLA, PEG-PLLLA, PEG-PDLLA, PEG-PDDLA, mPEG-PLA, mPEG-PLLLA, mPEG-PDLLA, mPEG-PDDLA, PEG-PCL, PEG-PLGA, PEG-PCL, mPEG-PCL, PEG-DPSE, mPEG-DPSE, PEO-PBD, mPEO-PBD, Pluronics (PEO-PPO-PEO), PLGA-PEG-PLGA, PEG-PLGA-PEG, PEG-PCL-PEG, PCL-PEG-PCL, Vitamin E-TPGS, Solutol HS15, and Soluplus, or a combination thereof.

In one aspect of the invention, a fatty acid is conjugated to the exterior surface of the nanoparticle to form a FA-NP. In another embodiment, two or more fatty acids are conjugated to the exterior surface of the nanoparticle to form a FA-NP. Any suitable fatty acid may be used to prepare the nanoparticles described herein. In some embodiments, the fatty acid conjugated to the exterior surface of the nanoparticle is a short-chain fatty acid (i.e., fewer than 6 carbon atoms in length). In some embodiments, the fatty acid conjugated to the exterior surface of the nanoparticle is an unsaturated fatty acid (e.g., oleic or erucic acid). In some embodiments, the fatty acid conjugated to the exterior surface of the nanoparticle comprises an aliphatic chain of between 1 and 30 carbon atoms. In some embodiments, the aliphatic chain comprises between 6 and 18 carbon atoms. In yet another embodiment, the aliphatic chain comprises between 6 and 12 carbon atoms. In some embodiments, the fatty acid is an omega-3, omega-6 or omega-9 fatty acid. In some embodiments, the omega-3 fatty acid is hexadecatrienoic acid, a-Linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid. In some embodiments, the omega-6 fatty acid is linoleic acid, gamma-linolenic acid, calendic acid, eicosadienoic acid, dihomoamma-linolenic acid, arachidonic acid, docosadienoic acid, adrenic acid, osbond acid, tetracosatetraenoic acid, and tetracosapentaenoic acid. In some embodiments, the omega-9 fatty acid is oleic acid, elaidic acid, gondoic acid, mead acid, erucic acid, nervonic acid, and ximenic acid.

In some aspects, the fatty is selected from the group consisting of lauric acid (C12), myristic acid (C14), palmitic acid (C16), steric acid (C18), alpha-linolenic acid (ALA), linoleic acid (LA), oleic acid (OA), docosahexaenoic acid (DHA), erucic acid (EA), formic acid, acetic acid, propionic acid, butyric acid, isobutyric acid, valeric acid, isovaleric acid and their derivatives that contain one long alkyl chain in which the number of carbon varies from 2 to 5, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, stearidonic acid, arachidonic acid and their derivatives that contain one long alkyl chain in which the number of carbon varies from 6 to 12, oxalic acid, malonic acid, succinic acid, glutaric acid, adipic acid, pimelic acid, suberic acid, azelaic acid, sebacic acid and their derivatives that contain one long alkyl chain in which the number of carbon varies from 8 to 20, citric acid, and tricarboxylic acid and its derivatives.

Any suitable method for conjugating the fatty acid to the exterior surface of the nanoparticle is contemplated by the invention. For example, conjugation may be achieved by means of chemical reaction or physical adsorption. In some embodiments, conjugation includes ionic, non-ionic, covalent or non-specific binding of the fatty acid to the exterior of the nanoparticle surface. In another aspect, conjugation of the fatty acid to the exterior surface of the nanoparticle includes adsorbing the fatty acid onto the surface of the nanoparticle, such as by spraying the nanoparticle with the fatty acid, followed by drying or in vacuo treatment such that the fatty acid is adsorbed into the surface of the nanoparticle. In some embodiments, fatty-acid conjugation to the exterior surface of the nanoparticle can be achieved as set forth in Example 1.

In some embodiments, the fatty acid is conjugated to the nanoparticle though the use of a solvent, such as acetone, acetonitrile (ACN), methanol (MeOH), tetrahydrofuran (THF), dimethyl sulfoxide (DMSO), or dimethylformamide (DMF), and the like. In some embodiments, oxalyl chloride (or other appropriate reagent for organic synthesis such as thionyl chloride, trichlorophosphine, trichlorophorous oxide, tribromophosphine, pentachlorophosphorane or other halogenated agent) is added to the fatty acid and solvent solution in dichloromethane (DCM) to form a first reaction mixture. In some embodiments, the first reaction mixture is maintained at 0° C. until such time that conversion into a corresponding fatty-acid chloride is required. In some embodiments, the first reaction mixture is subjected to first reaction conditions of one hour to 24 hours at room temperature. In some embodiments, a hindered organic base (such as trimethylamine, dimethylaminopyridine, di-isopropylethylamine, triethylamine (Et₃N), and the like) is added to the fatty-acid chloride, which is added to a solution of mono hydroxyl-functionalized PEG-b-PCL to form a second reaction mixture. In some embodiments, a second reaction is performed on the second reaction mixture for one hour to 24 hours at room temperature. In some embodiments, after the second reaction, the resulting mixture is concentrated in vacuo with the residue being dissolved in a minimal amount of DCM. In some embodiments, FA-NPs within the residue can be precipitated from the DCM with methanol, or other appropriate solvent.

In some embodiments, FA-NPs further comprise a therapeutic agent localized or encapsulated within the FA-NP. In one aspect, FA-NPs comprise a therapeutic agent within their core. In some embodiments, the therapeutic agent comprises a chemotherapeutic agent, antibiotic, antiviral drug, vaccine, diagnostic agent, monoclonal antibody or a binding fragment thereof, neuropeptide, CNS stimulant, anticonvulsant, antiemetic/anti-vertigo agent, muscle relaxant, narcotic analgesic, nonnarcotic analgesic, sedative, anti-inflammatory agent, cholinergic agonist, cholinesterase inhibitor, general anesthetic, addiction treatment drugs (such as alcohol-dependency), and an imaging agent.

In some embodiments, the therapeutic agent is an antibiotic that does not normally cross the BBB in an effective amount to treat a subject in need thereof in the absence of a delivery vector or modification. Examples of therapeutic agents that do not typically cross the BBB include, but are not limited to, first generation cephalosporin's (e.g., cephapirin, cephalothin, and cefazolin).

In one aspect, the therapeutic agent is an imaging agent such as a radio-contrasting agent (e.g., iodine or barium) or a detectable label. In some embodiments, the detectable label comprises a fluorescent label, dye, isotopic label, and the like. In one embodiment, the detectable label is selected from the group consisting of a radioactive label, a green fluorescent protein, a histag protein and P-galactosidase. In some embodiments, the imaging agent can be used as a sensor in a range of biological imaging applications such as PET, SPECT, MRI or fluorescence imaging. In some embodiments, the imaging agent can be used for cell labeling, cell staining, cell tracking, macrophage imaging and atherosclerosis imaging.

Other suitable therapeutic agents for delivery across the BBB using the FA-NPs of the invention include, but are not limited to, antibiotics (including, but not limited to, aminoglycosides, cephalosporins, quinolones, penicillins, tetracyclines, rifamycins, sulfonamides, anti-amoebic agents, and antifungal agents), antivirals (including, but not limited to, ganciclovir, acyclovir, and the like), steroids (including, but not limited to, dexamethasone, prednisolone, loteprednol, betamethasone, and the like), dilating agents (including, but not limited to, atropine, homatropine, cyclopentolate, and the like), non-steroidal anti-inflammatory agents (including, but not limited to, diclofenac, flurbiprofen, ketorolac, and the like), anti-metabolites (including, but not limited to, mitomycin C, 5-fluorouracil, and the like), anti-inflammatory agents (including but not limited to cyclosporins), and anti-VEGF agents such as bevacizumab and the like.

In some embodiments, the chemotherapeutic agent is aldesleukin (proleukin), altretamine (hexalen), amsacrine, ara-c cytarabine: cytarabine (ara-c), anastrazole, asparaginase, azacytidine, azidothymidine, carmustine, bendamustine, bevacizumab, bromocriptine, buserelin, busulfan, cabergolin, calcium folinate (leucovorin), camptosar (irinotecan), camptosar (irinotecan), capecitabine (xeloda), carboplatin (paraplatin), ccnu (lomustine), chloramucil (leukeran), cisplatin, cladribine (leustatin), clofarabine, cytosine arabinoside, cytarabine, cytoxin (cyclophosphamide), dacarbazine, dactinomycin, daunorubicin, decitibine, dexrazoxan, docetaxel (taxotere), doxorubicin hydrochloride (hydroxydaunorubicin), epirubicin, erlotinib (tarceva), estramustine, etoposide, exemestane (aromasin), fludarabine, fluorodeoxyuridine, 5-fluorouracil, flutamide, fulvestrant, gemcitabine, goserelin (zoladex), herceptin, hydroxyurea, idarubicin, ifosfamide, imatinib, interferon, ixempra (ixabepilone), lanvis thioguanine, lapatinib ditosylate (tykerb), lenalidomide (revlimid), letrozole (femara), luprone (luprolide), lomustine, lysodren, mechlorethamine hydrochloride, mitotan, megastrol, melphalan, mesna uromitexan, mercaptopurine, methotrexate, mitomycin, mitoxantrone, mitotane, navelbine vinerelobine, nelarabine, novladex, omustine, oxaliplatin, paclitaxel, panitumumab, paraplatin (carboplatin), patipilone epithilone b, pharmorubicin epirubicin, photofrin porfimer, pentostatin, procarbazine hydrochloride (natulan), trans-retinoic acid, rituxan (rituximab), somatuline lanreotide, streptozocin, sunitinib malate (sutent), tamoxifen, temodal temozolomide (temodar), teniposide, testosterone, topotecan, thioguanine, traztuzumab, thalidomide, thiotepa, tretinoin, vinblastine, vincristine, vepesid etoposide, vinorelbine, vindesine, vorinostat, or a combination thereof.

B. Properties of Fatty Acid Nanoparticles (FA-NPs)

In some embodiments, the FA-NPs described above have one or more of the following characteristics or beneficial properties. The FA-NPs of the invention comprise nanoparticles having a mean particle size of less than 1000 nm. In one aspect, the FA-NPs of the invention are preferably less than 200 nm in mean or average particle size.

It is to be understood that any sized nanoscale particle can be used in the invention. Nanoparticles can range in size from about 1 nm to about 500 nm in average mean particle size, about 1 nm to about 400 nm in average mean particle size, about 1 nm to about 300 nm in average mean particle size, about 1 nm to about 200 nm in average mean particle size, about 1 nm to about 100 nm in average mean particle size, and about 1 nm to about 50 nm in average mean particle size.

In other aspects, the size of the nanoparticles is from about 50 nm to about 500 nm (average mean particle size), from about 50 to about 400 nm, from about 50 nm to about 300 nm, from about 50 nm to about 200 nm, or from about 50 nm to about 100 nm.

In one embodiment, the nanoparticle has an average mean diameter of between 10 nm and 900 nm. In another embodiment, the nanoparticle has an average mean diameter of less than 200 nm. In yet another embodiment, the nanoparticle has an average mean diameter of about 25 nm to about 125 nm. In some embodiments, the nanoparticle is an ultrafine nanoparticle. In one embodiment, the nanoparticle is an ultrafine polymeric nanoparticle.

In another aspect, the FA-NPs disclosed herein comprise a narrow size distribution. In one embodiment, the Polydispersity Index (PDI) of the FA-NPs is less than 1.0. In some embodiments, the FA-NPs comprise a PDI of less than 0.5. In yet another embodiment, the FA-NPs disclosed herein comprise a PDI of less than 0.3. In some embodiments, the PDI of the FA-NPs is between about 0.1 and about 0.5. In another embodiment, the PDI of the FA-NPs is between about 0.2 and about 0.5. In yet another embodiment, the PDI of the FA-NPs is between about 0.2 and about 1.0.

In another aspect, the FA-NPs disclosed herein comprise a neutral charge at the nanoparticle exterior surface. Without being limited to the following, it is believed FA-NPs having a neutral charge allow for greater stability in an aqueous solution, for example by providing longer circulation times or enhanced accumulation of the FA-NPs at a site of interest in a subject.

In another aspect, the FA-NPs disclosed herein comprise a zeta potential of from about +/−30 mV to about +/−60 mV. In one embodiment, the zeta potential of the FA-NPs is greater than about +/−30 mV. In another embodiment, the FA-NPs zeta potential of the FA-NPs is greater than about +/−35 mV. In yet another embodiment, the FA-NPs comprise a zeta potential of from about +/−30 mV to about +/−40 mV.

In another aspect, the FA-NPs disclosed herein comprise a non-hemolytic and non-cytotoxic formulation. In some embodiments, cell viability after deliver of the FA-NPs is improved as compared to cell viability after delivery of the therapeutic agent alone. In one embodiment, the FA-NPs of the present invention improve the delivery of one or more therapeutic agents across the blood brain barrier. In some embodiments, the FA-NPs improve delivery of the therapeutic agent by a least 2-fold, 3-fold, 4-fold, 5-fold, 6-fold, 7-fold, 8-fold, 9-fold, 10-fold, 15-fold, 20-fold, 30-fold, 40-fold, 50-fold, or more. As a result, the FA-NPs of the present invention improve stability of the FA-NPs in blood plasma or serum as compared to stability of the therapeutic agent alone. In an exemplary embodiment, improved stability of a therapeutic agent within the FA-NPs can be measured by calculating circulation time of the therapeutic agent in the subject's circulatory system (e.g., blood). In one embodiment, improved stability of a therapeutic agent within the FA-NPs can be measured by calculating the time taken for half of the therapeutic agent dose administered to be eliminated from the subjects bloodstream (i.e., half-life). In some embodiments, the FA-NPs disclosed herein are prepared to form a micelle. In one embodiment, the FA-NPs undergo self-assembly to form micelle nanoparticles.

In some embodiments, the FA-NP further comprises two or more therapeutic agents localized or encapsulated within the FA-NP. In one embodiment, the therapeutic agent is a drug (such a cisplatin). Drug loading of FA-NPs produced according to any of the methods described herein is, in various embodiments, at least about 1%, at least about 5%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99.99%.

In one embodiment, the therapeutic agent within the FA-NP is present at a drug loading of between 1% and 99.99% (e.g., about 1% to about 99%, about 2% to about 98%, about 3% to about 95%, about 4% to about 90%, about 5% to about 80%, about 8% to about 75%, about 10% to about 60%, about 10% to about 50%, about 15% to about 50%, about 20% to about 50%). In another embodiment, the therapeutic agent within the FA-NP is present at a drug loading of between 5% and 50%. In yet another embodiment, the therapeutic agent within the FA-NP is present at a drug loading of between 15% and 60%. In some embodiments, the therapeutic agent within the FA-NP is present at a drug loading of between 10% and 80%.

In some aspects, the disclosure contemplates that the weight ratio of polymeric nanoparticle to therapeutic agent comprises from 10:1 to 1:10 as an initial or final weight ratio. In one embodiment, the weight ratio of polymer to therapeutic agent comprises from 5:1 to 1:5 as an initial or final weight ratio.

In some embodiments, the percentage of conjugation of fatty acid conjugated FA-NPs is between about 60% and 120%. In some embodiments the percentage of fatty acid conjugated FA-NPs is between 80% and 120%. In another embodiment, the percentage of fatty acid conjugated FA-NPs is between 90% and 100%.

In some embodiments, FA-NPs prepared according to any of the methods set forth herein, includes FA-NPs having constant or varied encapsulation efficiencies with respect to the therapeutic agent. Encapsulation efficiency of FA-NPs produced according to any of the methods described herein is, in various embodiments, at least about 1%, at least about 5%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, or at least about 99%.

In further embodiments, encapsulation efficiency of the therapeutic agent within the FA-NPs produced according to any of the methods described herein is from 1% to about 99%, or about 20% to about 90%, or about 30% to about 90%, or from about 40% to about 90%, or from about 10% to about 70%, or from about 10% to about 60%, or from about 10% to about 50%, or from about 20% to about 80%, or from about 20% to about 50%, or from about 5% to about 30%, or from about 1% to about 20%.

In one embodiment, the therapeutic agent within the FA-NP is present at an encapsulation efficiency of about 1% to about 99%. In another embodiment, the therapeutic agent within the FA-NP is present at an encapsulation concentration of between 10% and 60%. In yet another embodiment, the therapeutic agent within the FA-NP is present at an encapsulation efficiency of between 5% and 80%. In some embodiments, the therapeutic agent within the FA-NP is present at an encapsulation efficiency of between 10% and 30%.

In some embodiments, the therapeutic agent contained within or on the nanoparticle has a molecular weight of between about 50 Da and about 150 kDa. In some embodiments, the therapeutic agent contained within the nanoparticle has a molecular weight of about 50 Da to about 900 Da. In some embodiments, the therapeutic agent contained on or within the nanoparticle has a molecular weight of about 50 Da to about 500 Da.

In some embodiments, the therapeutic agent is released from the FA-NP over the course of days, weeks, or months. In some embodiments, the therapeutic agent is released from the FA-NP in a controlled release or extended release manner. The term “controlled release” as used herein, means that the therapeutic agent is released from the nanoparticle over a controlled or predetermined period or following a predetermined release profile. The term “extended release” as used herein means a formulation that provides for gradual release of the therapeutic agent over an extended period of time, and typically, although not necessarily, results in substantially constant blood levels of the therapeutic agent over an extended time period. In some embodiments, the controlled release profile or time course of release of a therapeutic agent may be modified by changing the ratio of FA-NPs to therapeutic agent, by changing the polymer or conjugation method used in the preparation of the FA-NPs, or by changing the porosity, pore size, or channel size of the FA-NPs, or by such other forms of manipulation and modification as are known to those skilled in the art. In one embodiment, the therapeutic agent is released such that the concentration of the therapeutic agent is effective to treat a subject in need thereof. In some embodiments, the therapeutic agent is released within the blood brain barrier such that the concentration of the therapeutic agent is effective to treat a CNS disorder in a subject in need thereof.

### II. Application of Fatty Acid Nanoparticles (FA-NPS)

Nanoparticles of the present invention are useful in the manufacture of a pharmaceutical composition or a medicament. In one embodiment, the FA-NPs described herein can be administered to a subject for the treatment of a central nervous disorder. In one aspect, the nanoparticles of the invention are formulated to target a site of interest. In one embodiment, the nanoparticles are formulated to traverse the blood brain barrier. In one embodiment, the FA-NPs are suitable for delivering a therapeutic agent across the blood brain barrier. In some embodiments, the FA-NPs of the invention having crossed the blood brain barrier affect one or more brain cells or cellular pathways therein. In some embodiments, the FA-NPs target delivery of the therapeutic agent across the blood brain barrier.

In one aspect, the FA-NPs described herein can be used to treat or diagnose a broad range of biological conditions. In one embodiment, the FA-NPs of the invention can be used to treat a central nervous disorder. In one aspect, a method for delivering a therapeutic agent comprises administering to a subject in need thereof of an effective amount of one or more of the FA-NPs disclosed herein. In one embodiment, the method includes treating a neurological disease such as Alzheimer's, Parkinson's or Huntingdon's disease. In another embodiment, the methods disclosed herein are suitable for the treatment of neurological diseases. In one embodiment, the neurological disease comprises a brain tumor, brain metastasis, schizophrenia, epilepsy, Alzheimer's disease, Parkinson's disease, Huntington's disease, dementia, inflammatory or infectious diseases of the central nervous system, stroke and blood-brain barrier related malfunctions.

In some embodiments, the therapeutic agent present within the FA-NPs includes, but is not limited to, a chemotherapeutic agent, antibiotic, antiviral drug, vaccine, diagnostic agent, monoclonal antibody or a binding fragment thereof, neuropeptide, CNS stimulant, anticonvulsant, antiemetic/anti-vertigo agent, muscle relaxant, narcotic analgesic, nonnarcotic analgesic, sedative, anti-inflammatory agents, cholinergic agonist, cholinesterase inhibitor, general anesthetic, and an imaging agent. For example, the therapeutic agent can comprise any suitable antibiotic. In one example, the antibiotic can be selected from the group consisting of cefotaxime, ceftizoxime, ceftriaxone, cefepime, vancomycin, metronidazole, sulfas, erythromycin, penicillin, amoxicillin, grepafloxacin, minocycline, levofloxacin, sparfloxacin, rifampin, cotrimoxazole and ciprofloxacin. In another embodiment, the therapeutic agent can comprise any suitable monoclonal antibody. In one example, the monoclonal antibody can be selected from the group consisting of 3F8, 8H9, Pritumumab, Ponezumab, Aducanumab, Bapineuzumab, Crenezumab, Gantenerumab, Erlizumab, and Refanezumab.

Depending on the application, the therapeutic agent may comprise two or more therapeutic agents, for example an antibiotic and an antiviral agent. For example, nanoparticles having an average particle size ranging from 1 nm to 1000 nm can act as suitable carriers for one or more therapeutic agents, preferably when the therapeutic agent(s) are smaller than the mean or average particle size of the FA-NPs.

The present invention can be administered to a subject in need thereof to treat central nervous system disorders. Such central nervous system disorders include but are not limited to Alzheimer's disease, Parkinson's disease, Huntingdon's disease, schizophrenia, epilepsy, stroke, traumatic brain injury, encephalitis, meningitis, depression, neuroblastomas, multiple sclerosis (MS), prion disease, amyotrophic lateral sclerosis (ALS), transverse myelitis, motor neuron disease, Pick's disease, Lyme disease, brain tumors, and spinal cord tumors.

The present invention can be administered to a subject in need thereof to diagnose central nervous disorders. In one embodiment, the FA-NPs of the present invention can comprise an imaging agent, such as a fluorescent dye (e.g., CLR1501 or CLR1502) that accumulates at a site of interest (e.g., brain tumor cells) that can be used to estimate the location, density, number, or presence of tumor cells at the site of interest.

### III. Administration of Fatty Acid-Conjugated Nanoparticles

The FA-NPs according to this invention for parenteral administration include sterile aqueous and non-aqueous solutions, suspensions, and emulsions. Injectable aqueous solutions contain the FA-NPs in water-soluble form. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. In some embodiments, the therapeutic agent to be localized or encapsulated within the FA-NP may also be in dried, e.g., lyophilized, form that may be rehydrated with a suitable vehicle immediately prior to encapsulation within the FA-NPs. In some embodiments, the FA-NPs disclosed herein are formulated for topical, oral, or parental administration.

In one aspect, the FA-NPs of the invention are formulated for injection (e.g., intravenous (i.v.,) intramuscular (i.m.,) subcutaneous (s.c.,)) as an aqueous solution. In one aspect, the FA-NPs disclosed herein are preferably administered via i.v. injection. In some embodiments, the FA-NPs comprise formulations for parenteral, intramuscular, subcutaneously, intranasal, intrathecal, intraparenchymal, intracerebroventricular, peroral, intracranial administration, and intraperitoneal administration.

In some embodiments, the FA-NPs as disclosed herein are formulated such that the therapeutic agent is released from the FA-NPs under controlled release or extended release conditions. In one aspect, the therapeutic agent is released from the FA-NPs over a course of days, weeks or months. In one aspect, the FA-NPs administered comprise biodegradable FA-NPs. In another embodiment, the FA-NPs administered comprise amphiphilic FA-NPs.

The FA-NPs disclosed herein can be administered to a subject at a therapeutically effective dose to treat or control a CNS disorder (e.g., brain tumor) as described herein. Typically, the FA-NPs are administered to a subject in an amount sufficient to elicit an effective therapeutic response in the subject.

The dosage of the therapeutic agent administered is dependent on the subject's body weight, age, individual condition, surface area or volume of the area to be treated and on the form of administration. The size of the dose also will be determined by the existence, nature, and extent of any adverse effects that accompany the administration of a particular therapeutic agent in a particular subject. For example, each therapeutic agent may have a unique dosage. Optimal dosing schedules can be calculated from measurements of therapeutic agent accumulation in the body of a subject. In general, dosage may be given once or more daily, weekly, or monthly. Persons of ordinary skill in the art can easily determine optimum dosages, dosing methodologies and repetition rates.

The term “dosage” as used herein, encompasses FA-NP formulations containing a therapeutic agent, optionally the therapeutic is located within the nanoparticle, expressed in terms of mg/kg/day or μg/kg/day. The dosage is the amount of the therapeutic agent administered in accordance with a particular dosage regimen. In some embodiments, the dosage is 50% of the “minimum approved dose”. As used herein, the minimum approved dose refers to the minimum dosage of a therapeutic agent that has received full regulatory approval by the appropriate regulatory authority (e.g., U.S. Food and Drug Administration (FDA) as safe and effective for human or veterinary use. In some embodiments, the dosage is 50%, 55%, 60%, 65%, 70%, 75%, 80%, 85%, 90%, 95% or more, of the minimum approved dose. In some embodiments, the FA-NPs can be administer at a dosage of between 10 μg/kg/day and 1 g/kg/day. In another embodiment, the FA-NPs can be administer at a dosage of between 50 μg/kg/day and 500 μg/kg/day. In yet another embodiment, the FA-NPs can be administer at a dosage of between 50 μg/kg/day and 300 μg/kg/day. In some embodiments, the FA-NPs can be administer at a dosage of between 10 μg/kg/day and 200 μg/kg/day. In another embodiment, the FA-NPs can be administer at a dosage of between 50 μg/kg/day and 800 μg/kg/day. In yet another embodiment, the FA-NPs can be administer at a dosage of between 100 μg/kg/day and 600 μg/kg/day. In a preferred embodiment, the FA-NPs are administered to a subject in need thereof via i.v. injection. In some embodiments, the frequency of i.v. injections is once, twice, or more a week; once, twice, or more a month; or once, twice, or more a month every month for a plurality of months (e.g., twice a month i.v. injections of the FA-NPs over the course of 6 months). In some embodiments, the dosage and/or frequency of FA-NPs administration is modified in accordance with the subject's response to administration of the therapeutic agent contained with the FA-NPs.

### EXAMPLES

The following examples are provided by way of illustration only and not by way of limitation. Those of skill in the art will readily recognize a variety of non-critical parameters that could be changed or modified to yield essentially the same or similar results.

### Example 1

### Preparation of Fatty Acid Conjugated Polymers

The objectives of this study were to (1) synthesize and prepare a library of FA-NPs; (2) prepare and characterize curcumin-loaded FA-NPs; (3) examine the in vitro toxicity of the FA-NPs; and (4) to demonstrate improved drug delivery to the brain in vivo.

### Data Analysis

All the experiments were conducted in at least triplicate, and results are expressed as mean±standard deviation. Unpaired Student t-tests were used to determine statistical significance; statistically significance was defined as p<0.05. Data analysis was conducted by using GraphPad Prism 5.

### Materials & Methods

### Synthesis of Fatty Acid Conjugated PEG-b-PCL, FA-PEG-b-PCL

We synthesized a library of fatty acid-conjugated PEG-b-PCL with nine different fatty acids (See, Table 1), namely, lauric acid (C12), myristic acid (C14), palmitic acid (C16), stearic acid (C18), alpha-linolenic acid (ALA), linoleic acid (LA), oleic acid (OA), docosahexaenoic acid (DHA) and erucic acid (EA), by using a convergent synthetic approach according to FIG. 1.

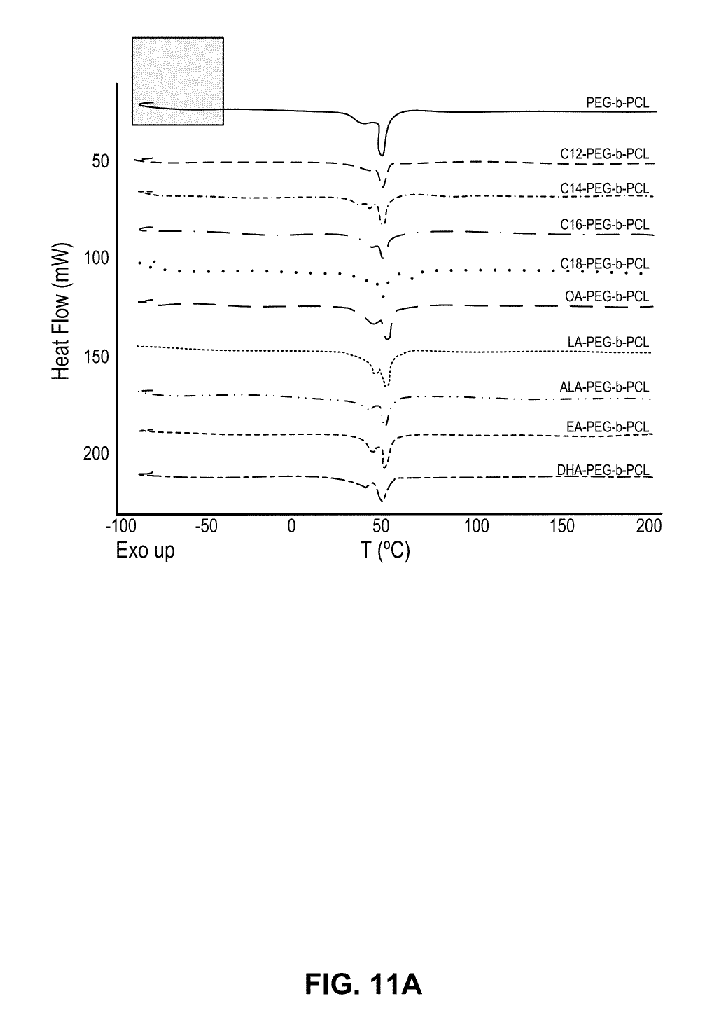

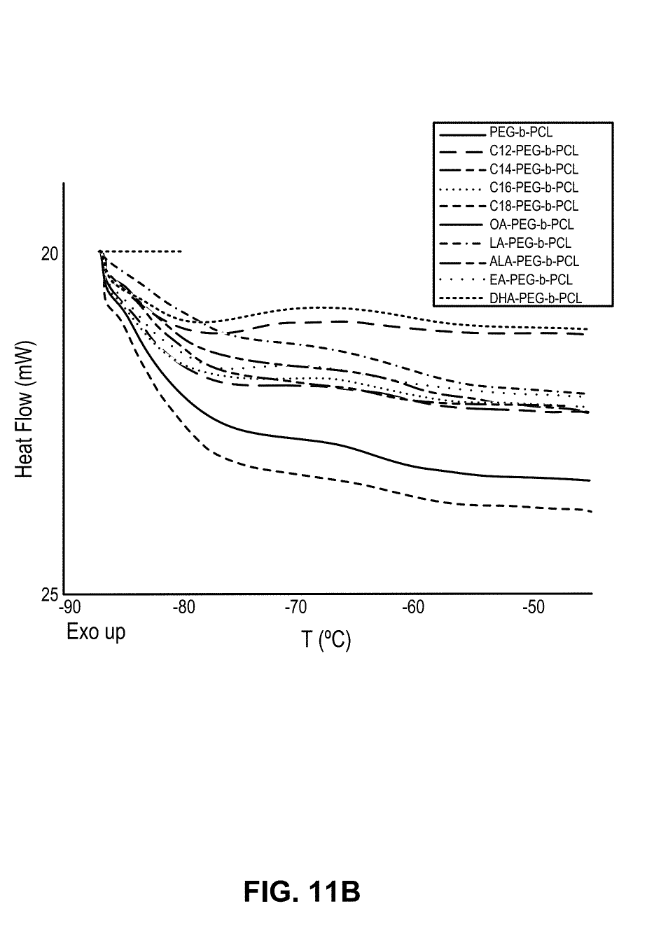

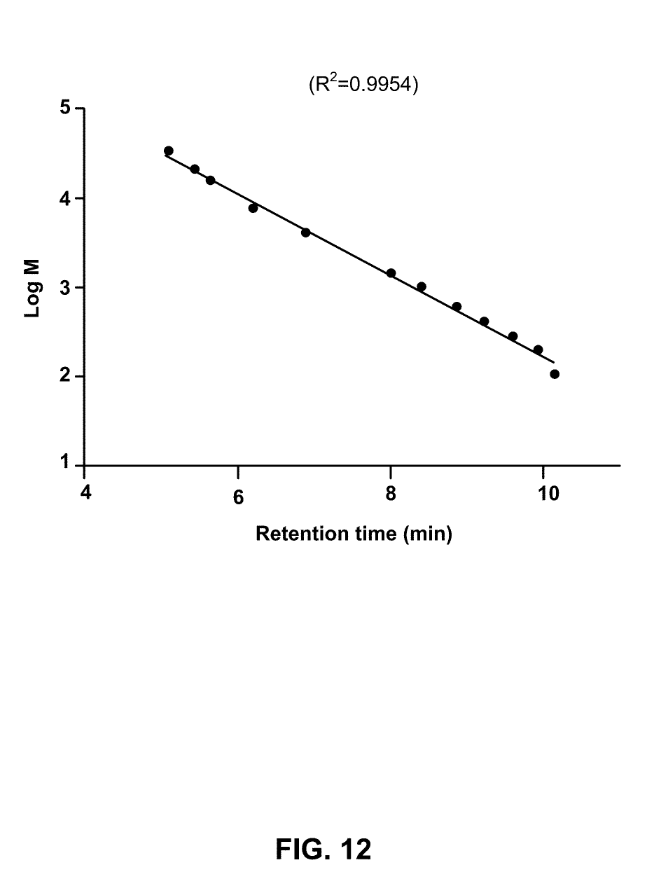

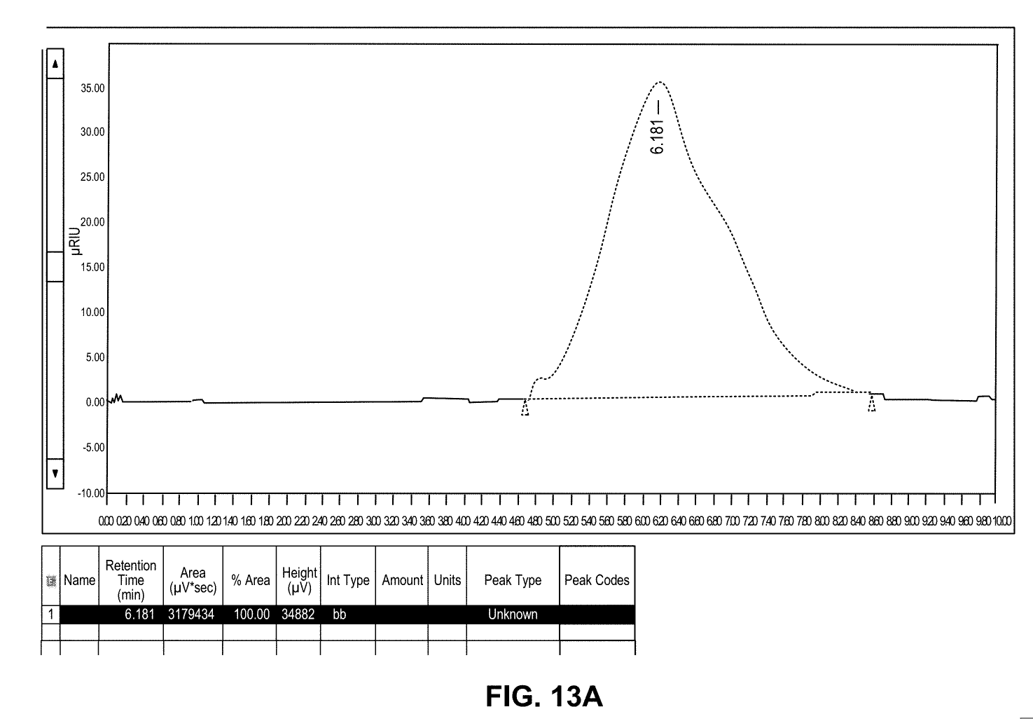

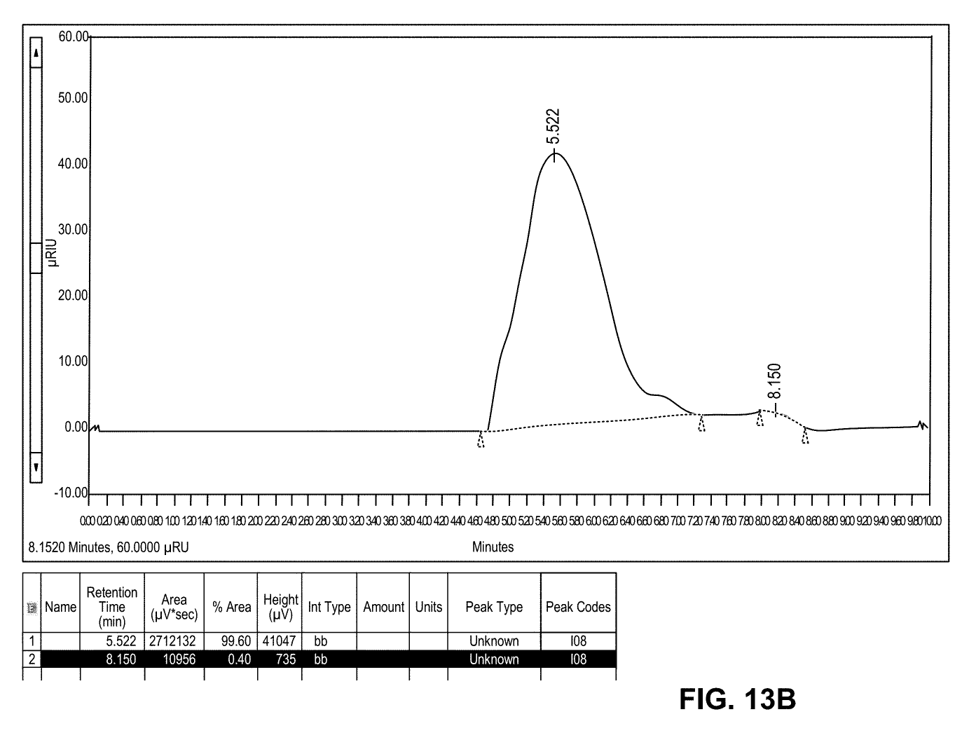

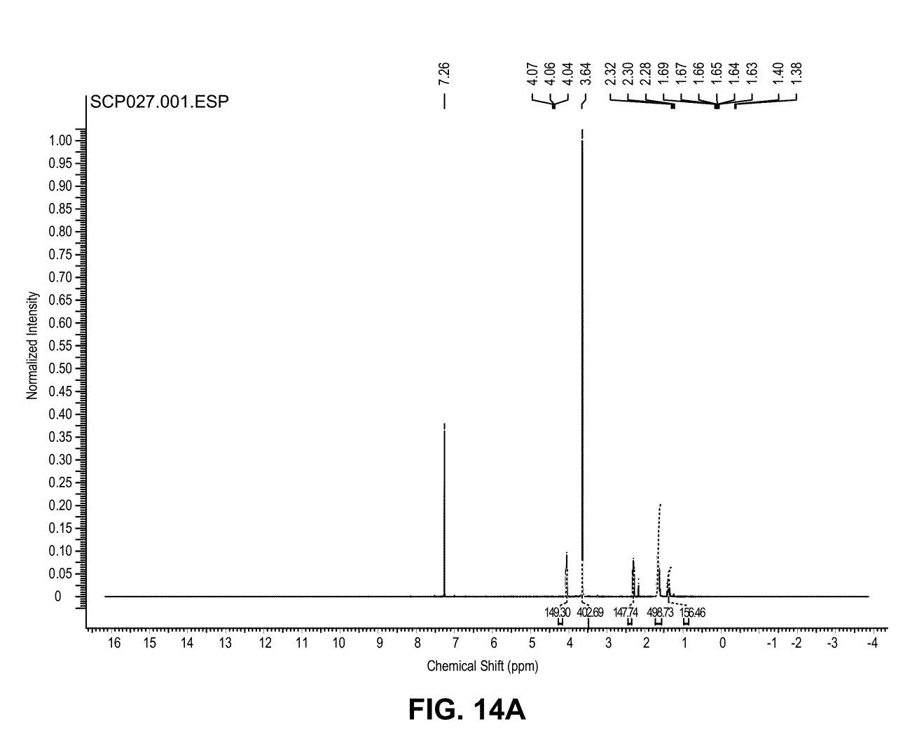

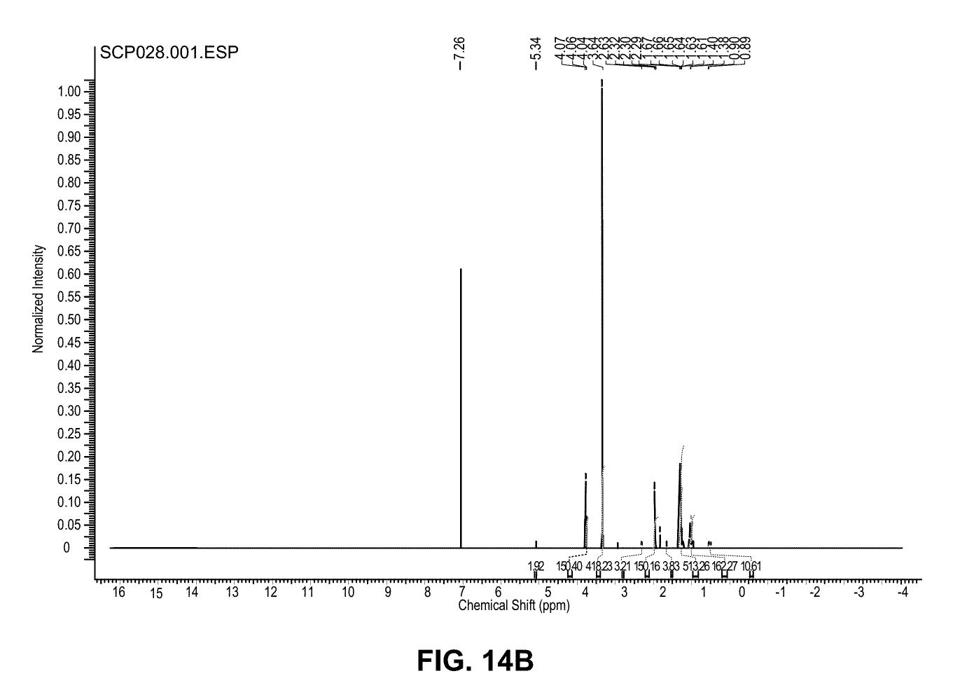

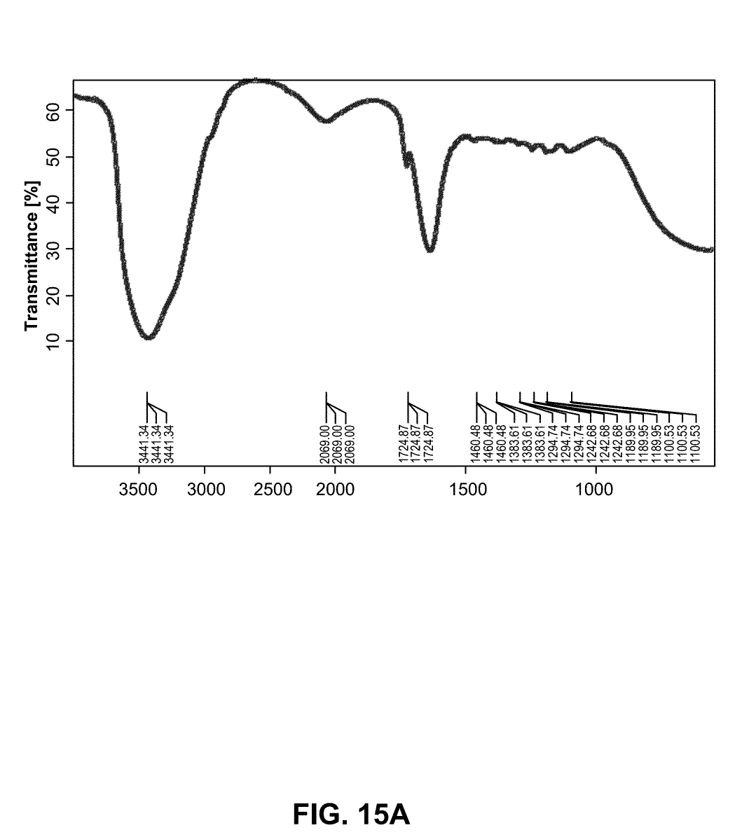

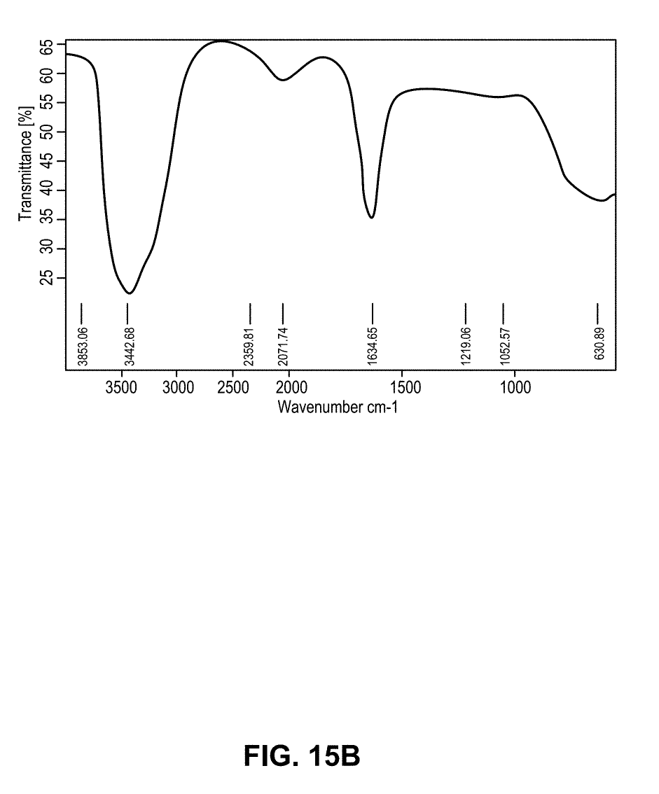

A catalytic amount of dimethylformamide (DMF) and oxalyl chloride (10 mmol, 10 equiv.) were added to a solution of a particular fatty acid (1 mmol) in dichloromethane (DCM) at 0° C. The reaction mixture was stirred at room temperature for 1 hr. After that, it was concentrated in vacuo to yield the corresponding fatty acid chloride and was used without further purification. Next, triethylamine (1 mmol) and the fatty acid chloride (1 mmol) were added to a solution of HO-PEG-b-PCL (0.1 mmol) in DCM (5 mL) at 0° C. The reaction mixture was subsequently stirred at room temperature for 16 hr. The reaction mixture was then concentrated in vacuo and the residue was dissolved in a minimum amount of DCM and the product was precipitated with MeOH. The percentage of conjugation was determined by ¹H NMR (FIG. 14A-14B) Further characterization of the FA-PEG-b-PCL was conducted by using fourier transform infrared spectrometry (FTIR) (FIG. 15A-15B), differential scanning calorimetry (DSC) (FIGS. 11A and 11B) and gel permeation chromatography (GPC) (FIG. 13A-13B). FIG. 11A shows a DSC chromatogram of FA-PEG-b-PCL from −87° C. to 200° C. FIG. 11B shows a DSC chromatogram of FA-PEG-b-PCL including an expanded region between −87° C. to −50° C.

### GPC Analysis of FA-PEG-b-PCL

The GPC analysis was conducted on a Waters ACQUITY UPLC H-Class System equipped with a styragel® HR3 column (300 mm×7.8 mm, 5 μm packing diameter) at 25° C. with a refractive index detector. The eluent employed was 100% THF at 1 mL/min flow rate. A calibration curve was constructed using polyethylene glycol standard (Polyethylene Glycol Easivials, Agilent Technologies). The peak molecular weight (Mp) of the standards is as follows: 34890, 21300, 16100, 7830, 4040, 1480, 1010, 610, 420, 282, 194, 106. The results are provided in FIGS. 13A-13B.

### Synthesis of Hydroxyl-Functionalized PEG-b-PCL, HO-PEG-b-PCL

HO-PEG-b-PCL was synthesized according to a method reported previously [8] using 1-pentanol as an initiator and polyethylene glycol (average Mₙ=4600) as the coupling block. Briefly, 1-pentanol was applied as an initiator in the ring-opening polymerization of ε-caprolactone to give hydroxyl functionalized polycaprolactone (PCL-OH), which was then reacted with succinic anhydride to yield carboxylic acid functionalized polycaprolactone (PCL-COOH). The PCL-COOH was subsequently coupled with polyethylene glycol (average Mₙ 4600) with a standard acyl chloride esterification method to give HO-PEG-b-PCL. The degree of polymerization of PCL was estimated to be 75 by ¹H NMR. Finally, HO-PEG-b-PCL was coupled to acyl chloride of a particular fatty acid to yield the corresponding fatty acid-conjugated PEG-b-PCL copolymer. The percentage of functionalization was by ¹H NMR.

TABLE 1
The molecular weight and percentage of conjugation of fatty acid
conjugated PEG-b-PCL.

| Polymer | % of conjugation^{a} | Mn^{a} | Mn^{b} | Mw^{b} | PDI^{c} |
|---|---|---|---|---|---|
| PEG-b-PCL | — | 13338 | 5710 | 9373 | 1.64 |
| C12-PEG-b-PCL | 94 | 13538 | 16908 | 19953 | 1.18 |
| C14-PEG-b-PCL | 106 | 13566 | 15902 | 18976 | 1.19 |
| C16-PEG-b-PCL | 86 | 13594 | 17153 | 20611 | 1.20 |
| C18-PEG-b-PCL | 116 | 13622 | 17370 | 20154 | 1.16 |
| ALA-PEG-b-PCL | 109 | 13586 | 17091 | 20817 | 1.22 |
| LA-PEG-b-PCL | 104 | 13588 | 17648 | 20735 | 1.17 |
| OA-PEG-b-PCL | 96 | 13590 | 13862 | 18202 | 1.31 |
| DHA-PEG-b-PCL | 62 | 13666 | 18362 | 22420 | 1.22 |
| EA-PEG-b-PCL | 101 | 13676 | 16141 | 19288 | 1.19 |

^{a}Determined by ¹H NMR.
^{b}Determined by GPC using PEG as the standard.
^{c}PDI = Mw/Mn, where M̅n = Σᵢ NᵢMᵢ/Σᵢ Nᵢ and M̅w = Σᵢ NᵢMᵢ²/Σᵢ NᵢMᵢ. where Nᵢ is the number of molecules of molecular mass Mᵢ.

### Example 2

### Preparation of Nanoparticles

Blank, curcumin-loaded NPs and coumarin-6 loaded NPs were prepared by a nanoprecipitation/anti-solvent method [9]. Briefly, various amount of a polymer was dissolved with or without a pre-defined amount of curcumin and coumarin-6 in a solvent (acetone, acetonitrile, tetrahydrofuran (THF), dimethylformamide (DMF) or dimethyl sulfoxide (DMSO)). The solution was subsequently added to 1 mL of type 1 water. For the samples prepared with acetone, methanol or THF, the solution was vortexed for 15 sec and placed in a vacuum concentrator for 20 min (45° C., 0.1 mbar) for the removal of the volatile organic solvent. For DMF or DMSO, the resulting solution was dialyzed against type 1 water for 1 day. After removing the organic solvent, the resulting solution was subjected to centrifugation at 16,000 g for 3 min to remove any un-encapsulated curcumin. The supernatant was collected and used in our studies and characterization.

### Optimization of the Preparation of Nanoparticles.

Nanoprecipitation is a commonly used method for the preparation of polymeric nanoparticles due to its simplicity. Multiple factors need to be considered for the proper selection of solvents for this process. This method requires the use of two solvents. The first one is usually an organic solvent to dissolve polymers and drugs together and this solution will be added to another miscible solvent, usually aqueous solvents such as water, buffer, etc., which acts as a poor (anti-solvent) solvent for both the polymers and the drugs. The organic solvent will be subsequently removed. Typically, volatile organic solvents such as acetone, acetonitrile, methanol etc., are preferred owing to their easy removal under reduced pressure. However, some poorly soluble drugs cannot dissolve in these volatile organic solvents at sufficient concentrations. In this case, some “magic solvents” such as DMSO, DMF, NMP, etc., can be used. These solvents are non-volatile and their removal has to be accomplished by dialysis, ultrafiltration, etc.

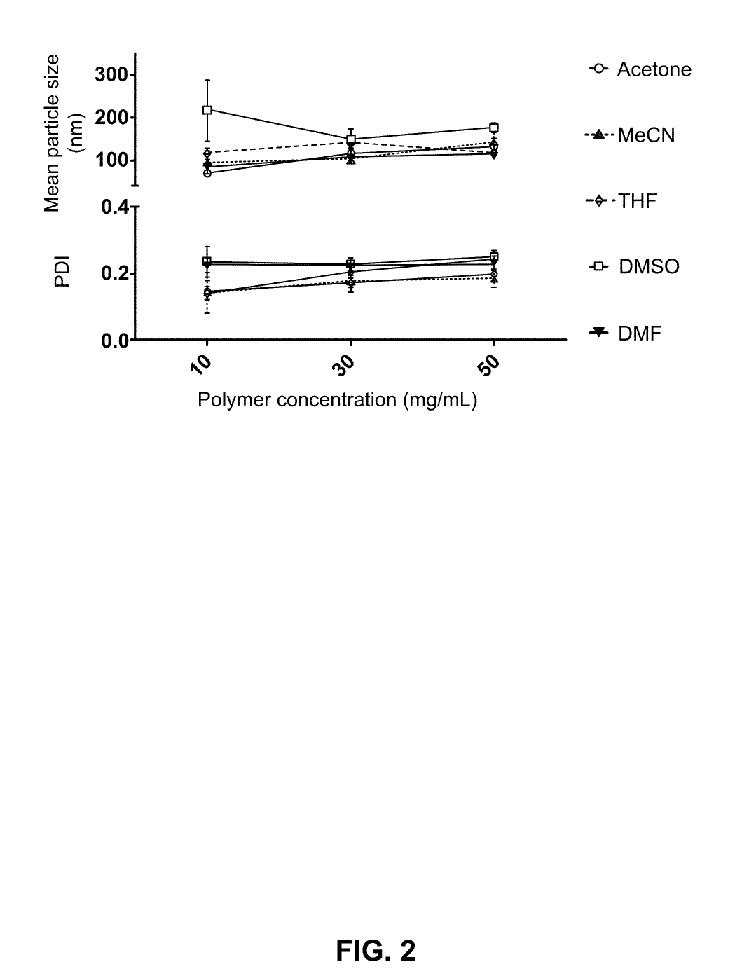

PEG-b-PCL was used to optimize the nanoprecipitation process for the encapsulation of curcumin. Since both PEG-b-PCL and FA-PEG-b-PCL share the same solubilizing core, it is expected that their encapsulation efficiency is comparable. Five different solvents (acetone, THF, acetonitrile, DMF and DMSO) and three polymer concentrations (10, 30 and 50 mg/mL) were used to investigate their influence on the average particle size and polydispersity index (PDI) of the NPs. The effect of solvent and polymer concentration on the particle size and PDI of blank PEG-NPs are summarized in FIG. 2.

A general trend was observed that the mean particle size and PDI of the NPs increased with polymer concentrations and this phenomenon is in excellent coherence with the reported studies [11]. Polymeric NPs tend to aggregate at high concentrations, leading to an increase in particles size and PDI. Apart from the polymer concentrations, the solvents employed also had a significant impact on the particle size and PDI of the NPs. DMSO, a poor solvent for the solubilization of PCL, yielded very large particle size (>150 nm) and PDI (>0.2). Other volatile solvents like acetone (69 nm), acetonitrile (94 nm) and THF (115 nm) were able to produce NPs of different size and narrow PDI. DMF, a non-volatile solvent, was able to produce NPs (83 nm) but the PDI was marginally higher (0.23). Our results demonstrate the versatility of the nanoprecipitation method in controlling the particle size of NPs via a proper selection of the organic phase, polymer concentration, etc.

In addition to the particle size and PDI, solvent selection also plays a significant role in the encapsulation of drugs into NPs. In this study, curcumin was used as a model to investigate the loading capacity of our NPs at 10 mg/mL polymer concentration. The extremely low water solubility of curcumin often restricts the application of this herbal compound in medical applications [7]. Both acetone (Table 2, entry 4) and THF (Table 2, entry 7) yielded high drug loading and the presence of curcumin did not influence the particle size and PDI of the NPs significantly. Attempts to further increase the drug loading led to poor physical stability with curcumin precipitation within hours (Table 2, entry 1-3 and 5-6). However, the low drug loading resulted from the use of DMF may be due to a different method employed for the removal of the organic solvent. DMF is non-volatile and therefore dialysis was applied to the solvent elimination. Drug molecules may have diffused out of the dialysis bag, yielding a low drug loading and this is indeed the case in our study. Yet, substantial improvement in the solubility of curcumin was still observed.

TABLE 2
Solvent effect on the curcumin loading in PEG-NPs at 10 mg/mL polymer
concentration (polymer concentration = 10 mg/mL).

| Entry | Solvent | DL (%)^{a} | [Curcumin] (mg/mL) | Δ_{size}^{b} | Stability^{c} |
|---|---|---|---|---|---|
| 1 | acetone | 25.36 | 3.40 ± 0.39 | 1.18 | * |
| 2 | acetone | 23.39 | 3.04 ± 0.19 | 1.13 | * |
| 3 | acetone | 19.24 | 2.38 ± 0.20 | 1.09 | ** |
| 4 | acetone | 16.03 | 1.91 ± 0.20 | 1.04 | *** |
| 5 | THF | 26.08 | 3.56 ± 0.76 | 0.96 | * |
| 6 | THF | 21.68 | 2.77 ± 0.20 | 1.06 | ** |
| 7 | THF | 18.43 | 2.26 ± 0.14 | 1.20 | *** |
| 8 | MeCN | 11.33 | 1.28 ± 0.31 | 0.89 | ** |
| 9 | MeCN | 9.96 | 1.06 ± 0.32 | 0.84 | ** |
| 10 | MeCN | 5.09 | 0.54 ± 0.05 | 0.90 | ** |
| 11 | DMF | 3.53 | 0.37 ± 0.27 | 0.69 | *** |

^{a}Drug loading (DL %).
^{b}mean particle size (curcumin loaded)/mean particle size (blank).
^{c}Stability of the curcumin loaded PEG-NPs judged visually at 30 min after removal of the organic solvent; *** Clear solution without precipitation ** Cloudy suspension * Heavily precipitated.

Taken together, nanoprecipitation employing acetone as the organic solvent at a polymer concentration of 10 mg/mL appeared to be the best condition because it yields high drug loading and stability, and the particle size and PDI are within an acceptable range. This condition was applied to prepare NPs for subsequent studies.

### Example 3

### Characterization of Nanoparticles

Nine different FA-PEG-b-PCL polymers with different fatty acids were used to encapsulate curcumin based on the method described above. The results of which are summarized in Table 3.

TABLE 3
Physical properties of curcumin-loaded nanoparticles (polymer
concentration = 10 mg/mL).

| | Particle size (nm)^{a} | PDI^{a} | DL (%)^{b} | EE (%)^{c} | Zeta Potential (mV)^{a} |
|---|---|---|---|---|---|
| PEG-NPs | 79.99 ± 4.87 | 0.14 ± 0.04 | 16.03 | 95.42 | −12.21 ± 2.54 |
| C12-NPs | 151.43 ± 2.18 | 0.22 ± 0.03 | 14.09 | 82.03 | −4.88 ± 0.91 |
| C14-NPs | 48.03 ± 6.64 | 0.19 ± 0.06 | 15.50 | 91.70 | 0.22 ± 1.60 |
| C16-NPs | 77.67 ± 8.02 | 0.27 ± 0.02 | 16.23 | 96.86 | −1.16 ± 0.13 |
| C18-NPs | 253.78 ± 195.13 | 0.21 ± 0.08 | 3.69 | 19.17 | 0.50 ± 0.32 |
| ALA-NPs | 44.27 ± 1.14 | 0.19 ± 0.01 | 17.02 | 102.57 | 0.92 ± 0.43 |
| LA-NPs | 45.58 ± 1.65 | 0.13 ± 0.03 | 16.63 | 99.75 | 2.86 ± 0.27 |
| OA-NPs | 45.93 ± 1.34 | 0.19 ± 0.02 | 16.26 | 97.08 | −16.43 ± 0.25 |
| DNA-NPs | 46.91 ± 1.11 | 0.19 ± 0.02 | 16.39 | 98.00 | −1.14 ± 2.47 |
| EA-NPs | 50.01 ± 5.39 | 0.22 ± 0.03 | 15.96 | 94.94 | −12.85 ± 0.96 |

^{a}Determined by DLS.
^{b}DL = Drug loading.
^{c}EE = encapsulation efficiency.

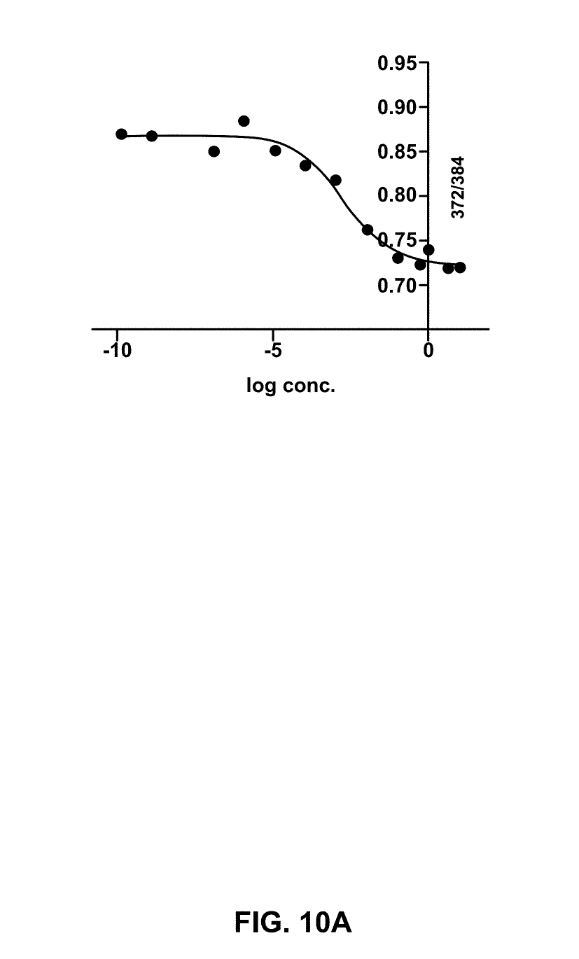

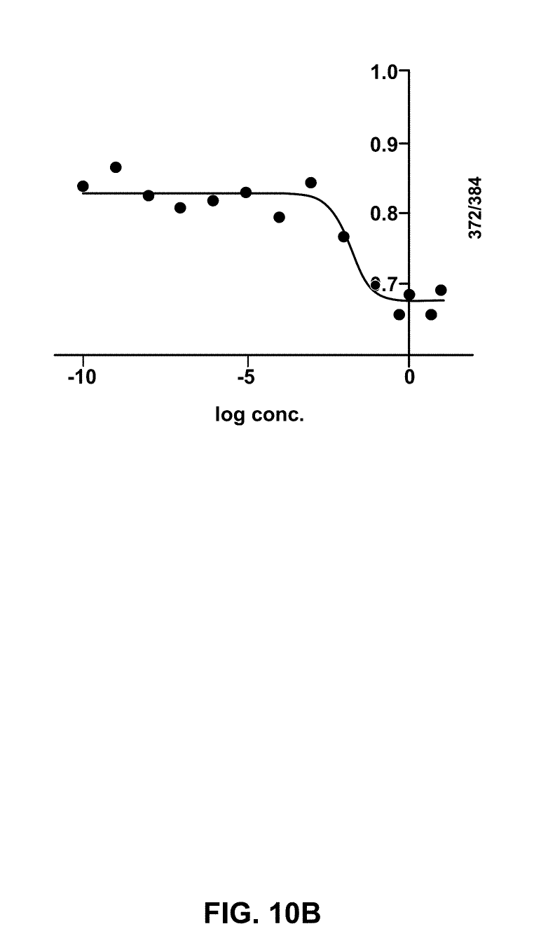

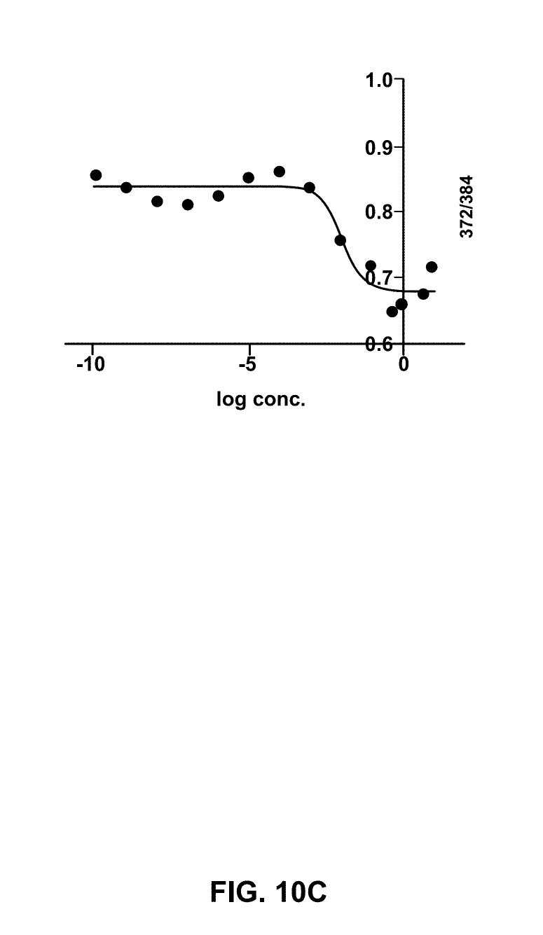

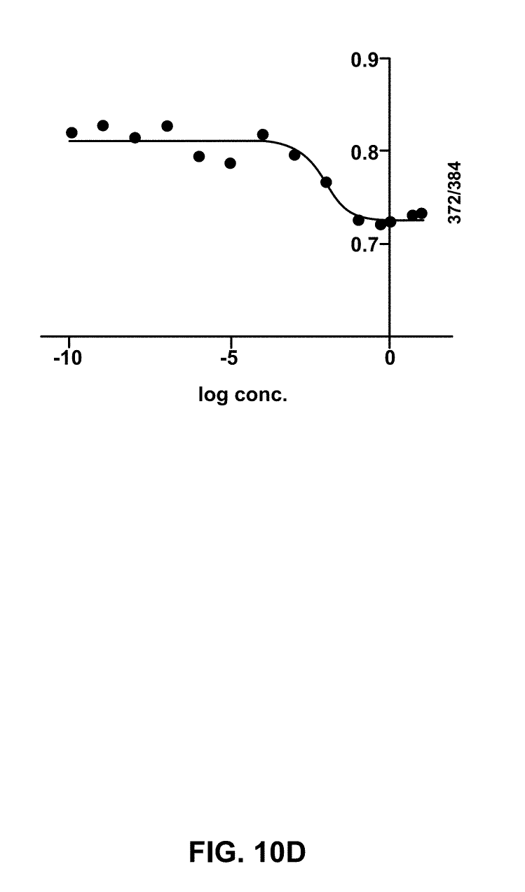

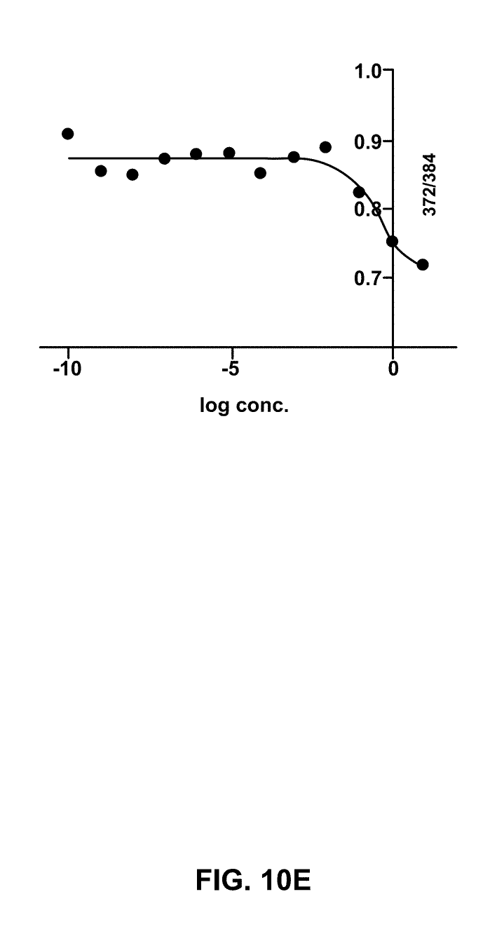

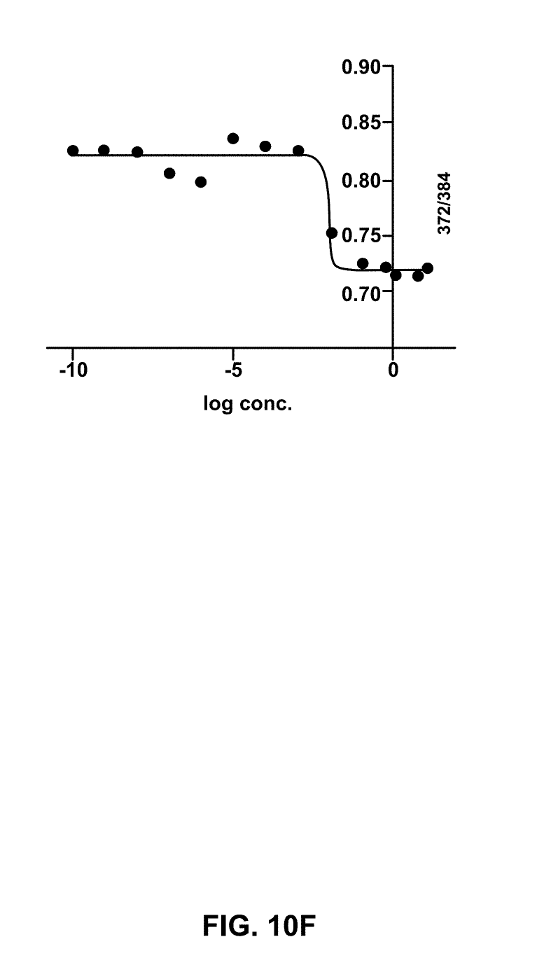

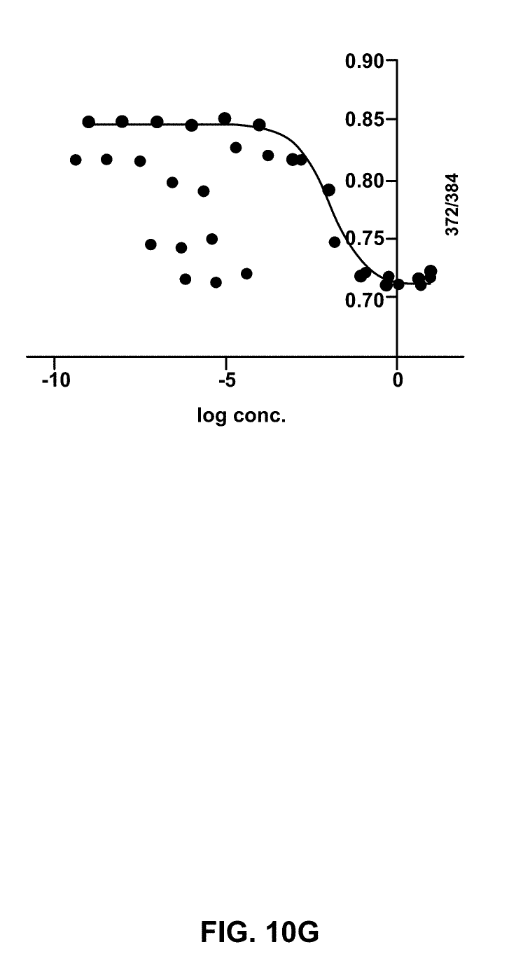

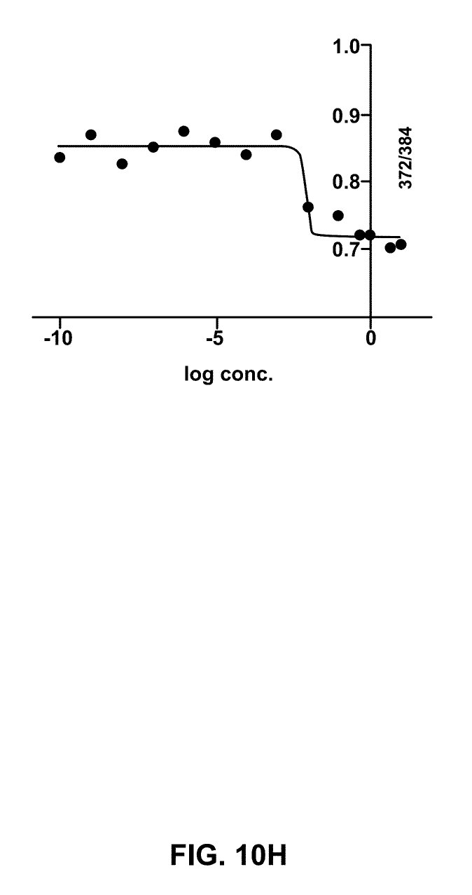

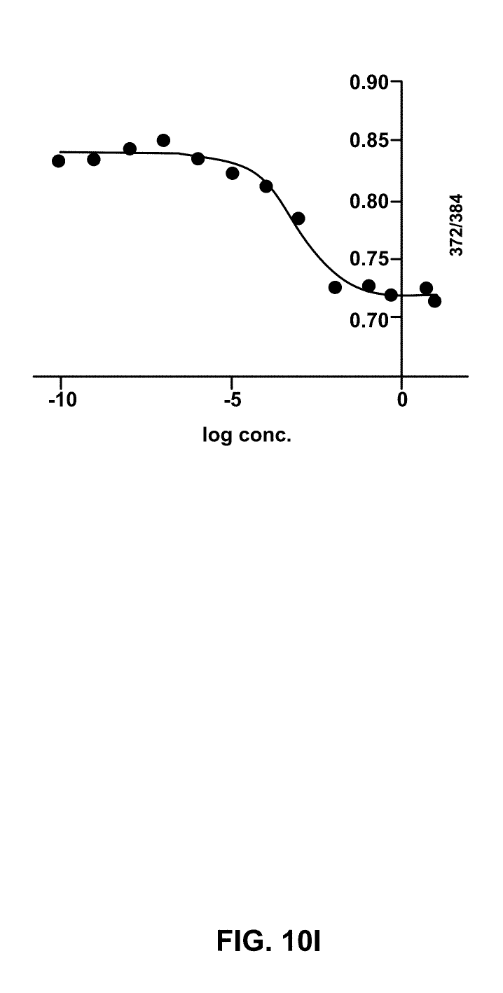

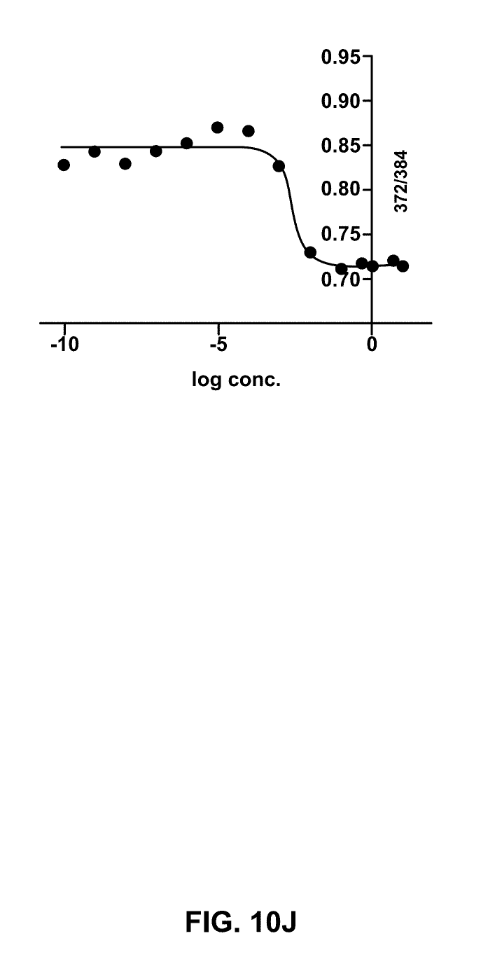

The CMC of blank FA-NPs was determined to be 0.8 to 16 μg/mL (See, FIG. 10A), comparable to the reported value in the literature for polymeric micelles of PEG-b-PCL [11].

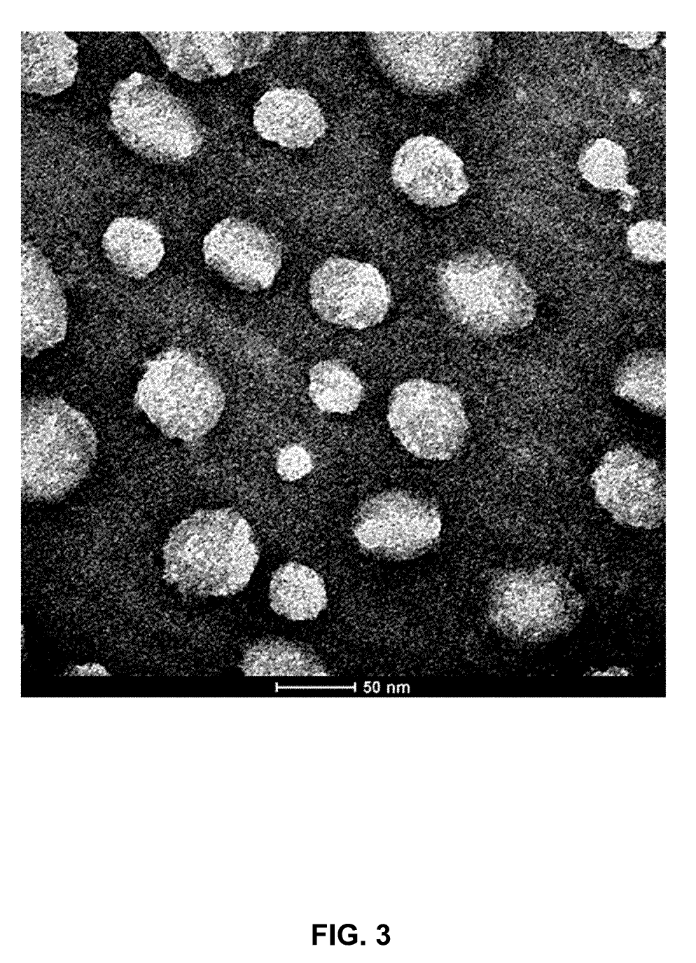

The zeta potentials of FA-NPs were determined to be approximately ±30 mV, indicating a fairly neutral charge at the nanoparticle surface. This is not unexpected due to utilization of the carboxylic group of the fatty acids in the conjugation process. In addition, TEM images were also captured for oleate acid (OA)-NPs (100 μg/mL polymer concentration), indicating the formulation of spherical nanoparticles (FIG. 3).

With respect to the drug loading and encapsulation efficiency, NPs with different fatty acids showed similar drug loading (14 to 17%) and encapsulation efficiency (>80%) except C18-NPs, and this may be attributed to the poor formation of the NPs from C18-conjugated PEG-b-PCL, as revealed from the abnormal particle size and PDI (data not shown). The particle size was the only physical parameter which was significantly altered by the fatty acids, but it was still within a narrow range (from 45 to 80 nm) except C12- and C18-NPs. NPs of PEG-b-PCL conjugated with unsaturated fatty acids such as ALA, LA, OA, DHA and EA showed very narrow particle size range, averaging around 44 to 50 nm (see, Table 3). For the saturated FA-NPs, the particle size appears to vary with the chain length of the fatty acids, however, an explicit correlation was not observed.

### Determination of Drug Concentration

The concentration of curcumin and coumarin 6 in each formulation were determined by a UPLC/UV method. The UPLC/UV analysis was conducted on an Agilent 1290 equipped with a BDS Hypersil C18 column (250 mm×4.6 mm, 5μm packing diameter) at 25° C. The eluents employed were A: 0.1% v/v solution of formic acid in water and B: acetonitrile at 2 mL/min flow rate. The method, retention time and detection wavelength are summarized below:

| | | Retention | |
|---|---|---|---|
| | | time | Wavelength |
| | Method | (min) | (nm) |
| Curcumin | 0 to 4 min (50% B). 4 to 5 min | 4.62 | 420 |
| | (50% to 80% B). 5 to | | |
| | 7 min (80% B). | | |
| Coumarin 6 | 0 to 2 min (50% B). 2 to 3 min | 6.47 | 446 |
| | (50 to 80% B). 3 to | | |
| | 7 min (80% B). | | |

The calibration curves were linear over the concentration range of (curcumin) 0.32 to 200 μg/mL (R²=1.0000), 8 to 1000 μg/mL (R²=0.9950) and (coumarin 6) 0.32 to 200 μg/mL (R²=1.0000). All the intra-day and inter-day precision (RSD) of all QC sample (10 or 200 μg/mL) was within 2% and the accuracy was within 90 to 110%.

Drug loading and encapsulation efficiency were calculated by the following equations:

$$DL ( \% ) = \frac{weight of the drug in supernatant}{weight of the drug and polymer added} \times 100 \%$$

$$EE ( \% ) = \frac{weight of the drug in supernatant}{weight of the drug added} \times 100 \%$$

### Physical Characterization of Nanoparticles

The cumulative particle size (Z-Average mean particle size, Dz), polydispersity (PDI) and zeta potentials of blank and curcumin loaded nanoparticles were analyzed by dynamic light scattering (DLS) with Delsa™ Nano C (Beckman coulter, Brea, Calif., USA) equipped with a 658 nm laser source.

### Transmission Electron Microscopy (TEM) of Oleate-Conjugated PEG-b-PCL NPs (OA-NPs)

The morphology of OA-NPs was captured by using TEM with a negative staining method (FIG. 3). After dilution to 100 μg/mL polymer concentration, the sample was deposited on a carbon-coated copper grid (200 mesh) for 30 min. Excess fluid was drawn off followed by staining with a 4% uranyl acetate solution for 1 min and allowed to dry before analysis (Tecnai G2 Spirit, FEI, Oreg., USA).

### Determination of Critical Micelle Concentration (CMC)

The CMCs of FA-NPs were determined by using a pyrene absorption method [10]. Briefly, 10 μL of 20 μM pyrene in 1% ethanol was added to 90 μL of polymer solutions at different concentrations. The fluorescence intensity at 373 nm (I1) and 384 nm (I3) was measured. A plot of 11/13 versus log (polymer concentration) using a 4-parameter non-linear curve fit was used to determine the CMC of the micelles. For non-ionic micelles, the center of the sigmoid represents the CMC. The results of these experiments are shown in FIGS. 10A-10J.

### Release Profile of Drug-Loaded Nanoparticles

The in vitro release profiles of the drug-loaded NPs were assessed in 0.1% Tween 20 at 37° C. based on a dialysis method reported earlier with modifications [11]. Briefly, 1 mL of the NPs was added into a dialysis tube (Float-A-lyzer G2 with a membrane MWCO 3.5-5 kD, Spectrum Laboratories, Inc., Rancho Dominguez, Calif., USA), which was subsequently placed in 250 mL of 0.1% Tween 20 at 37° C. Dialysate was replaced periodically. Samples (10 μL) were withdrawn from the dialysis tube at designated time points. The samples collected were analyzed using a UPLC-UV method and GraphPad Prism 5 was employed to plot the cumulative drug release versus time. The diffusional behaviour of curcumin (2 mg/mL in isopropanol) was also determined using the same procedure, except the dialysate was replaced with isopropanol.

### Stability of Nanoparticles in Serum

An advantage of applying NPs in drug delivery is the prolongation of the circulation time and alteration of the biodistribution profiles of drug molecules in vivo. To achieve this, the drug molecules must be retained inside the nanoparticles. A Förster resonance energy transfer (FRET) experiment was applied to estimate the stability of NPs in serum.[12]

DiO was used as a donor fluorophore (λₑₓ: 484 nm and λₑₘ: 508 nm) and DiI was used as an acceptor fluorophore (λₑₘ: 508 nm and λₑₘ: 570 nm) in our study. When both FRET pairs are encapsulated inside the same nanoparticle and excited at the donor fluorophore wavelength, energy transfer will occur due to the close proximity of the two dyes (DiO/DiI Förster radius: 45 A), resulting in a very strong emission at 570 nm (acceptor emission) [12]. By contrast, when NPs disintegrate or the dyes leak out, such energy transfer will be significantly depressed because of the increased distance between the FRET pairs, leading to a shift in the emission peak from 570 to 508 nm. Therefore, by monitoring the FRET ratio (508/570 nm), the stability of NPs in serum can be estimated.

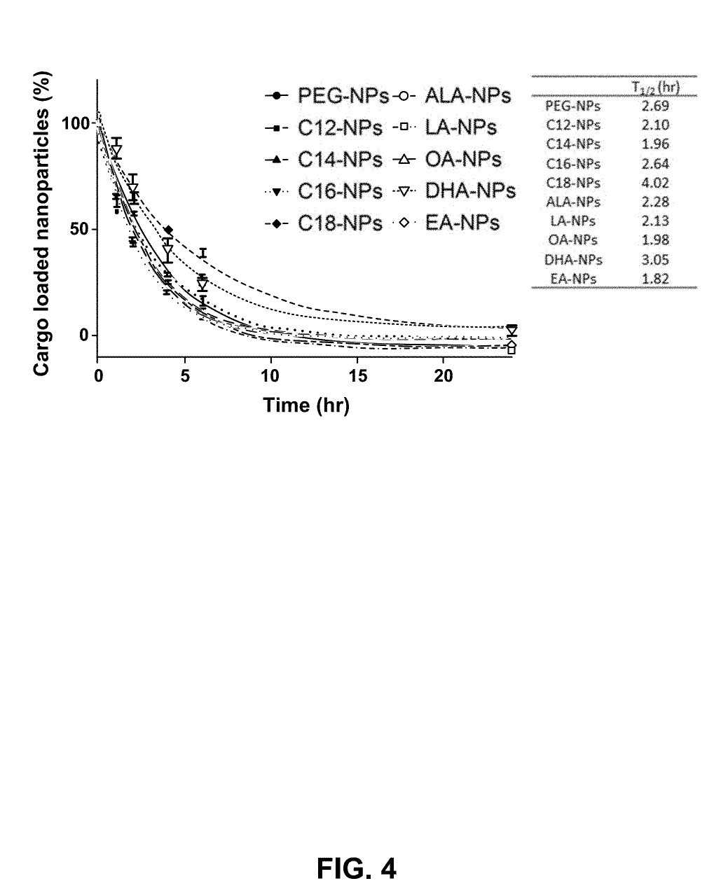

The stability of curcumin-loaded NPs in serum was estimated using Förster Resonance Energy Transfer (FRET) fluorescent dye-loaded NPs, based on a reported method [12, 13]. The fluorescent intensity at λ₁ (507 nm) and λ₂ (580 nm) was monitored for 2 days. The ratio of λ₁/λ₂ was used to estimate the percentage of intact NPs in the sample. The ratio at T=0 represents 100% intact NPs; 0% intact NPs was achieved by incubation of the nanoparticles with serum for 48 hours followed by quenching with 0.1% Triton X-100. The stability of DiO/DiI-loaded nanoparticles in serum after 24 hr is provided in FIG. 4. The initial polymer concentration employed was 1 mg/mL. The polymer to serum ratio is equal to 1:9; which gives a final polymer concentration of 100 μg/mL. The half-lives of the nanoparticles were estimated using an one phase exponential decay equation from GraphPad Prism 5.

At t=0 hr, the FRET ratio is defined as 100% intact NPs; and at t=48 and after addition of Triton X-100, the FRET ratio is defined as 0% intact NPs. The curve was fitted using an one phase exponential decay equation with Prism 5 (GraphPad Software, Inc., California, USA). The final concentration of each polymer used in this experiment was 100 μg/mL in a medium with a protein concentration around 32-70 mg/mL.

Generally speaking, in the blood, the plasma proteins adsorb non-specifically onto the surface of NPs, causing de-stabilization and premature drug release. Typically, the PEG corona of NPs can resist this protein adsorption and stabilize the NPs. The half-life and PEG-NPs was determined to be 2.69 hr. The incorporation of fatty acids on the surface of the NPs did not significantly alter their half-lives (Unpaired student t-test, p>0.05). With different fatty acid on the surface of NPs, their half-lives range from 1.96-4.02 hrs, which is fairly narrow. In addition, a clear “structure-activity relationship” of the fatty acids on the half-lives of the corresponding nanoparticles cannot be explicitly established.

### In Vitro Release Studies of Curcumin Loaded Nanoparticles

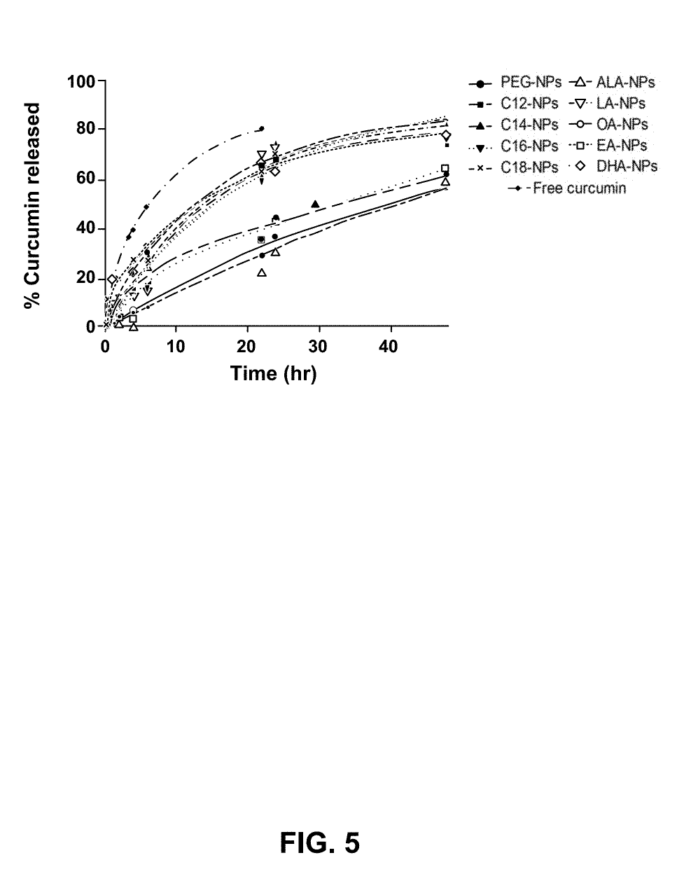

In order to fully utilize the targeting ability of FA-NPs, the loaded cargo (i.e., therapeutic agent, such as a drug) must be retained inside the NPs with controlled release profiles. Otherwise, premature drug release may result in drug precipitation, rapid clearance from the circulation and/or systemic side effects. Compared with free curcumin solution which completely diffused out of the dialysis tube within 24 hours (>80% released), curcumin-loaded NPs sustained the drug release for at least two days, which is typical for polymeric micelles (FIG. 5). The curcumin concentration of each nanoparticle was at the optima loading (described in Table 3). The curcumin concentration for the free curcumin is 2 mg/mL dissolved in 2-propanol.

### Toxicity of Nanoparticles

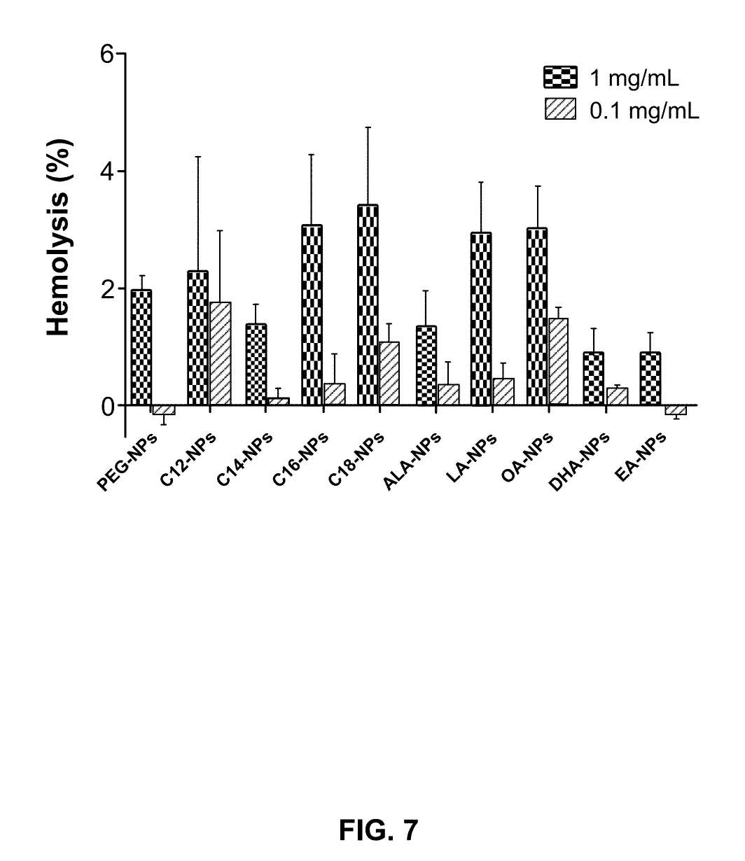

In addition to the physical properties, safety is another important element for the application of FA-NPs for drug delivery to the brain. Here, we assessed the FA-NPs for hemolysis and cytotoxicity. FA-NPs appear to be non-hemolytic according to the ASTM E2524-08 standard (<5%), indicating the safe nature of these NPs for intravenous administration (FIG. 7). Our studies are in line with reported studies that high molecular weight MPEG-b-PCL nanoparticles (i.e. PEG: Mₙ≥2000 and PCL: Mₙ>3500) showed insignificant hemolytic activity [15]. The results indicate that the presence of fatty acids does not significantly increase the hemolytic activity of PEG-b-PCL. By contrast, fatty acid-conjugated polysorbate (e.g. TWEEN® 80) exhibit substantially higher hemolytic toxicity (>10% at 8 μM, 10 μg/mL) [16].

Mouse brain endothelial (bEnd.3) and human neuroblastoma (SH—SY5Y) cell lines were used to estimate the toxicity of FA-NPs to the BBB and neuron cells, respectively. A mouse brain endothelial cell line (bEnd. 3), and a human neuroblastoma cell line (SH—SY5Y), were obtained from ATCC (Virginia, USA). bEnd. 3 cells were cultured in DMEM with 10% FBS and SH—SY5Y cells were cultured in 1:1 mixture of MEM and DMEM-F12 with 10% FBS, at 37° C. with 5% CO₂ according to the ATCC guidelines. Cells with a passage number between 30 and 35 were used in this study.

### Cytotoxicity

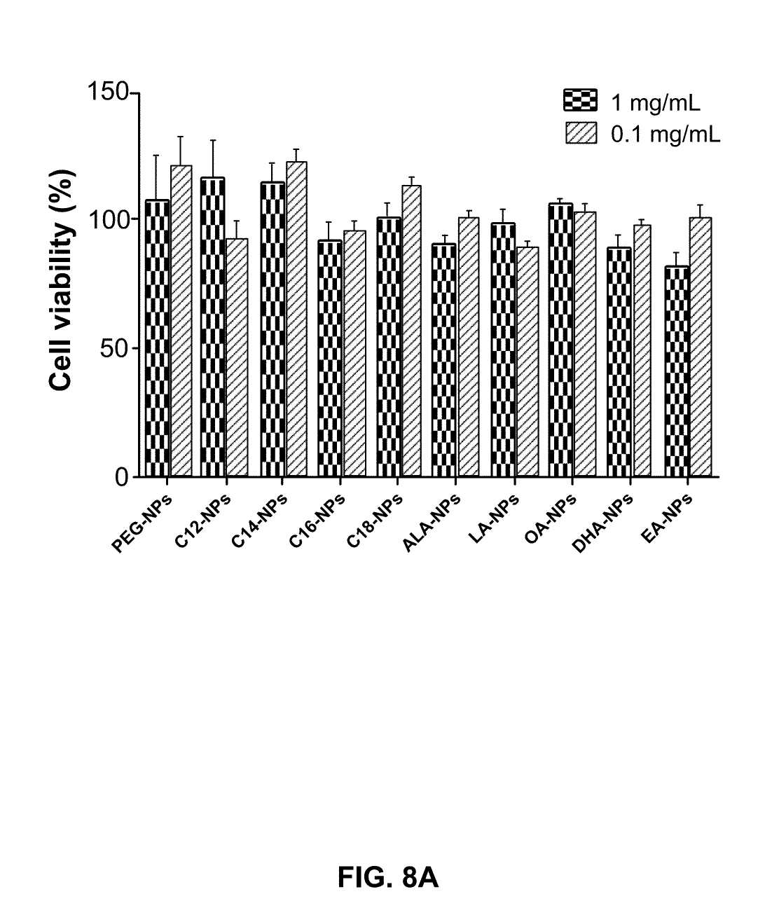

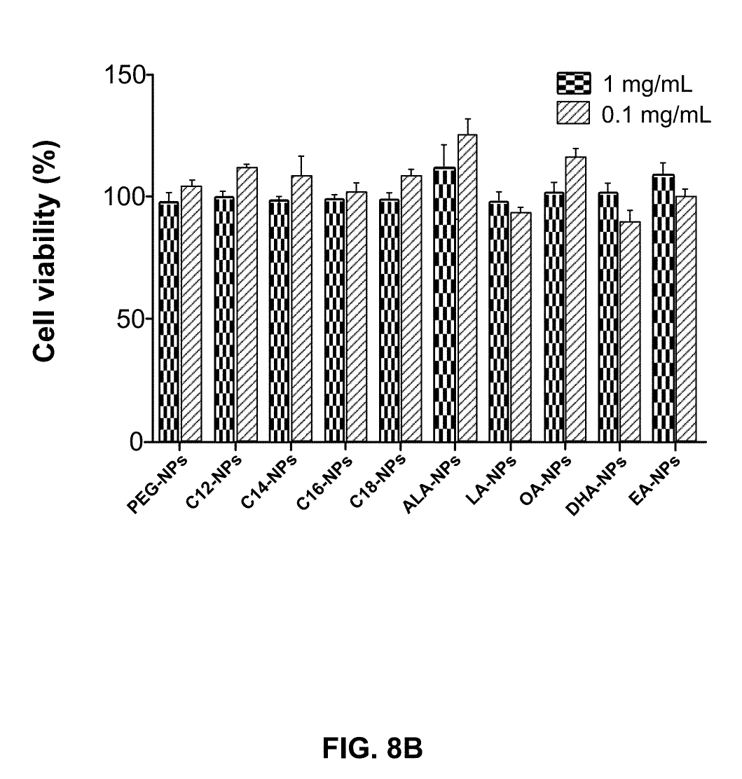

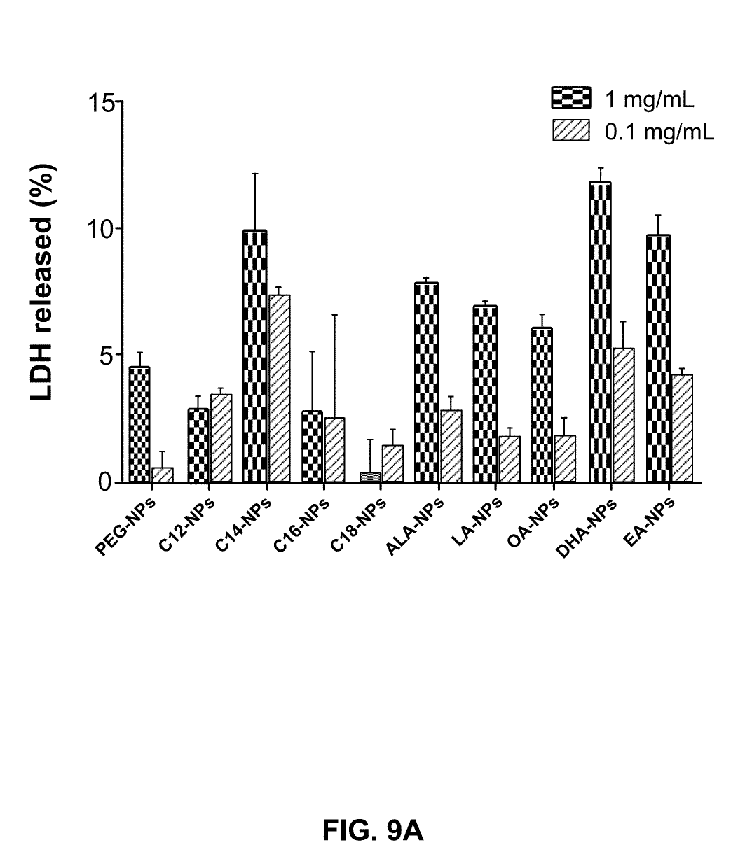

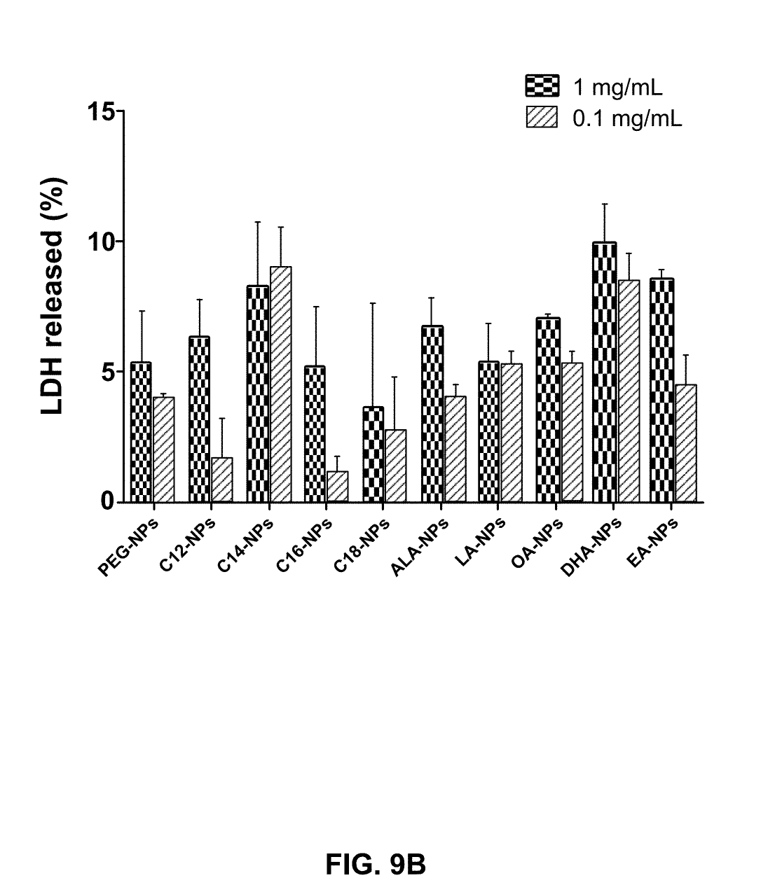

5×10³ (2×10⁴ for MTT assay) of bEnd. 3 or 2×10⁴ SH—SY5Y cells per well were seeded in a 96-well plate and cultured in the corresponding cell culture media for 24 hr. The cell culture media were subsequently removed and the cells were washed three times with PBS. Next, 100 of blank NPs diluted in the cell culture media at different concentration was added to the cells, which were incubated for 1 day. After that, the cytotoxicity of each formulation to the cells was assessed using MTT and LDH assays, according to the manufacturer guidelines. The effect of the nanoparticles on the viability of bEnd.3 cells or SH—SY5Y cells determined by MTT assays are summarized in FIGS. 8A and 8B, respectively. The effect of the nanoparticles to the viability of bEnd.3 cells or SH—SY5Y cells determined by LDH assays are summarized in FIGS. 9A and 9B, respectively.

The equations for determining the cell viability and LDH released are as follows:

$$Cell viability ( MTT , measured at 470 nm ) = \frac{{abs}_{treated} - {abs}_{no cell}}{{abs}_{no treatment control} - {abs}_{no cell}} \times 100 \%$$

$$LDH released ( LDH , measured at 490 nm ) = \frac{{abs}_{treated} - {abs}_{no treatment control}}{{abs}_{lysed control} - {abs}_{no treatment control}} \times 100 \%$$

The cell membrane-damaging effect of these NPs were revealed by LDH assays. LDH is an enzyme staying in the cytoplasm and LDH cannot leave cells unless the cell membrane is damaged. It is a well-known fact that most surfactant based NPs induce cytotoxicity via membrane damage [17]. In the bEnd.3 and SH—SY5Y cell models (FIGS. 8A, 8B, 9A and 9B), dose-depending toxicity was observed in most of the NPs (except in C12-NPs and C18-NPs in bEnd.3 and C14-NPs and LA-NPs in SH—SY5Y, in which comparable toxicity was observed at different polymer concentrations). In general, FA-NPs caused higher LDH release than PEG-NPs. Even so, the LDH release caused by most of the NPs at 1 mg/mL was generally less than 10% (except DHA in bEnd.3 cells) and it was less than 10% at 0.1 mg/mL polymer concentration for all the nanoparticles. The amount of LDH release is considered to be minimal, indicating that the NPs cause negligible cell membrane damage. MTT assays were also applied to assess the cytotoxicity of the NPs by measuring the mitochondria activity. The cell viability did not exhibit any dose-dependency and was generally greater than 90% for all the NPs at both 0.1 and lmg/mL polymer concentrations in both cell lines (FIGS. 8A, 8B, 9A and 9B). Taken together, FA-NPs appear to be safe with minimal cytotoxicity and hemolytic activity.

### Hemolysis Assay

Hemolysis assay was performed using fresh heparinized blood from Sprague-Dawley rats. The blood was subjected to centrifugation at 800 G for 5 min. The supernatant was discarded and the pellet (erythrocytes) was collected. The stock dispersion was prepared by replacing blood plasma in PBS and was stored at 2 to 8° C. until use. 10 μL of NPs was added to 90 μL of the stock dispersion. PBS was used as a negative control and 10% SDS with 0.5 mM EDTA solution was used as a positive control. The solutions were mixed and incubated at 37° C. for 1 hr on an orbital shaker (800 rpm). The solution was then subjected to centrifugation at 800 G for 5 min. Supernatant was collected and the percentage of hemolysis was determined by measuring UV absorbance at 420 nm and the data was normalized against the positive control. The results of these experiments is summarized in FIG. 7. Cell viability was greater than 90% for all the NPs at both 0.1 and lmg/mL polymer concentrations in both cell lines (data not shown). Taken together, FA-NPs appear to be safe with minimal cytotoxicity and hemolytic activity.

### EXAMPLE 4

### In Vivo Brain Accumulation of Coumarin 6-Loaded Nano Particles

We selected OA-NPs, LA-NPs, ALA-NPs, C16-NPs and DHA-NPs as models for assessing the in vivo brain accumulation of FA-NPs, which were assessed in rats after intravenous injection. In this study, coumarin 6 was employed as a surrogate for curcumin due to the intense fluorescent signal of this dye for easy detection and the brain uptake of this dye from seven different formulations, 0.1% DMSO solution, PEG-NPs, OA-NPs, C16-NP, LA-NP, ALA-NP, and DHA-NP was studied.

All the animal studies followed the guidelines issued by the Department of Health, Hong Kong and the Animal Experimental Ethics Committee (AEEC) at the Chinese University of Hong Kong.

Male Sprague-Dawley® rats with a weight between 220 to 250 g were supplied by the Laboratory Animal Services Centre, The Chinese University of Hong Kong. The rats were housed at a 12/12 hour light/dark cycle with free access to water and standard laboratory chow. Coumarin 6 -loaded NPs (30 mg/mL polymer concentration and 171 μM of coumarin 6) and coumarin 6 in 1% DMSO were injected into the rats through the left lateral tail vein (bolus injection, 136 μg/kg). After designated time point, the rats were sacrificed by exsanguination via cardiac puncture after CO₂ anesthesia. Blood was then collected, heparinized and centrifuged at 1000 G for 5 min for plasma collection. Brain, was collected after perfusion with cold 0.9% saline through the left ventricle (approx. 500 mL per rat). Organs were homogenized in saline. Coumarin 6 was extracted from the organ homogenate and plasma samples with acetonitrile. The concentrations of coumarin 6 in the extracts were quantified using a fluorescent microplate reader (Clariostar®, BMG Labtech, Germany) (Ex470-15 nm, Em507-15 nm, dichroic filter 485 nm). By spiking coumarin 6 standard solutions into blank organ homogenates/plasma before extraction, the percentage of recovery was determined to be >95%.

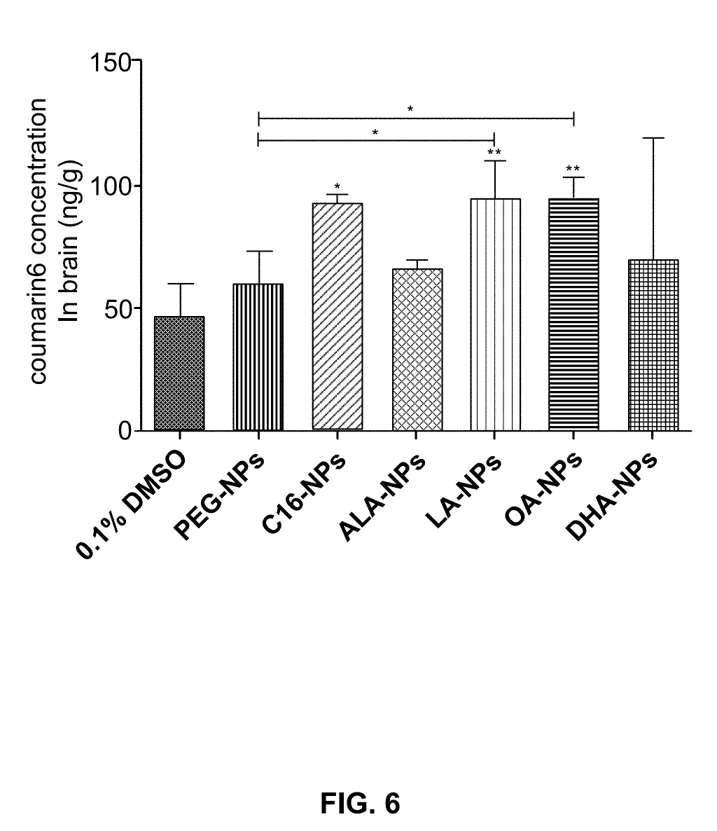

The results of this experiment are summarized in FIG. 6 which shows the concentration of coumarin-6 in rat brain, 30 minutes after intravenous injection of each different coumarin 6 formulation at a dosage of 136 μg/kg (n=3). The data in FIG. 6 is reported as mean±SD. One-way ANOVA followed by Dunnett's multiple comparison test against PEG-NPs and 0.1% DMSO. Statistical significant was defined as: p>0.05 =not significance (ns), p≤0.05=*, p≤0.01=**, p≤0.001 =***, p≤0.0001=****. Annotation above the capped line indicates statistical test against PEG-NPs. Annotation above the column indicates statistical test against 0.1% DMSO.

In this experiment, both OA-NPs (93.64±9.82 ng/g) and LA-NPs (94.20±15.94 ng/g) shows significant higher coumarin 6 concentration in the brain, as determined using one-way ANOVA followed by Dunnett's multiple comparison test, than PEG-NPs (58.61±14.45 ng/g). The improvement in the concentration of coumarin 6 in the brain can be explained by the fact that both oleic acid and linoleic acid are substrates of FATPs. Oleic acid, a monounsaturated omega-9 fatty acid with a lipid number of 18:1 cis-9, is a known substrate for FATP-1 and FATP-4. Linoleic acid, a polyunsaturated omega-6 fatty acid with a lipid number of 18:2 cis-9,12, is a known substrate for FATP-4. Other ligands such as palmitic acid, a known substrate for FATP-4, only demonstrated non-significant improvement than PEG-NPs, but was significantly better than 0.1% DMSO. Ligands such as alpha-linolenic acid and docosahexaenoic acid, known substrates for mFABP-5, demonstrated no improvement over PEG-NPs or 0.1% DMSO.

### Example 5

### Cellular Uptake of Coumarin 6-Loaded Nanoparticles

In this experiment, in vitro cellular uptake of coumarin 6 loaded nanoparticles were evaluated to investigate the uptake mechanism of OA-NPs.

Here, cellular uptake of coumarin 6-loaded nanoparticles and mechanistic study. 2×10⁴ cells per well were seeded in a 96 well plate and cultured in DMEM with 10% FBS for 1 day. The medium was removed and the cells were washed three times with PBS. For the uptake study, 100 μL of coumarin 6-loaded NPs in DMEM was added and the well-plate was incubated for 2 hours. For the uptake mechanism study, 50 μL of chemical inhibitor was added and the well-plate was incubated for 1 hour, followed by the addition of 50 μL of coumarin 6-loaded NPs in DMEM. After incubation for 4 hours, the formulations were removed and the cells were washed three times with PBS. The fluorescent intensity was quantified by using a fluorescent microscope (Nikon Eclipse Ti, Nikon, Japan).

### Immunofluorescent Staining of FA-NPs

2×10⁵ bEnd.3 cells per well were seeded in a 24 well plate and cultured in DMEM with 10% FBS for 1 day. The medium was removed and the cells were washed three times with PBS. Then 100 μL of FA-NPs or PEG-NPs (5% w/w 5-carboxyfluorescein content) in DMEM was added and the well-plate was incubated for 2 hours. After that the cells was washed three times with PBS, then fixed using 10% neutral-buffered formalin for 10 minutes at room temperature. After washing the cells three times with PBS, it was blocked with 1% BSA, 22.52 mg/mL glycine in PBST (PBS with 0.1% Tween 20) for 30 minutes. Cells was then incubated with the primary antibody (10 μg/mL) in 1% BSA in PBST for 1 hour at room temperature. After washing the cells three times with PBS, a secondary antibody (20 μg/mL) in 1% BSA was added and the cells were incubated for 1 hour at room temperature in the dark. Then, cells were washed with PBS 3 times and the corresponding pictures were captured by using a fluorescent microscope (Nikon Eclipse Ti, Nikon, Japan).

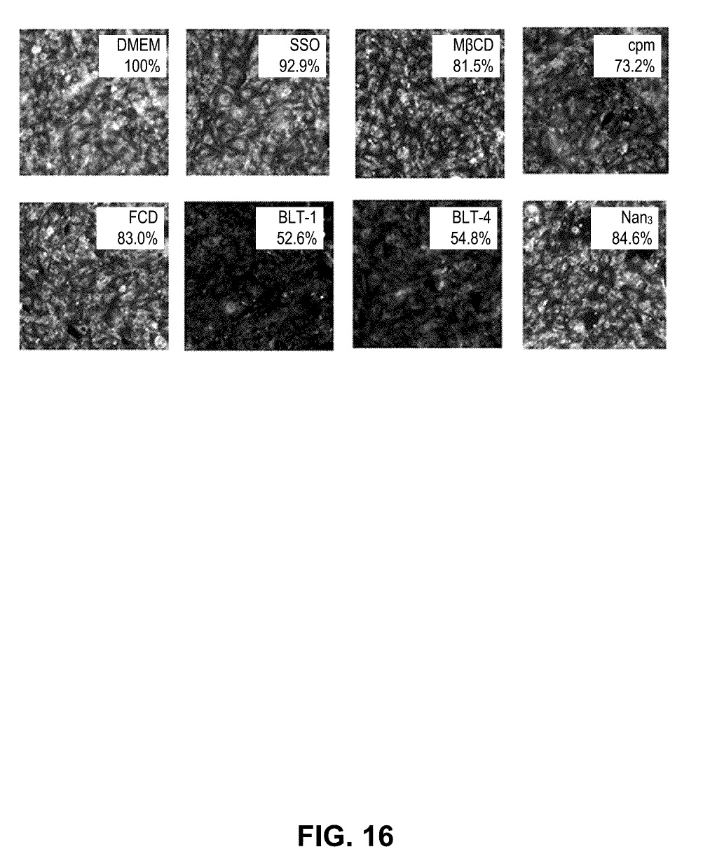

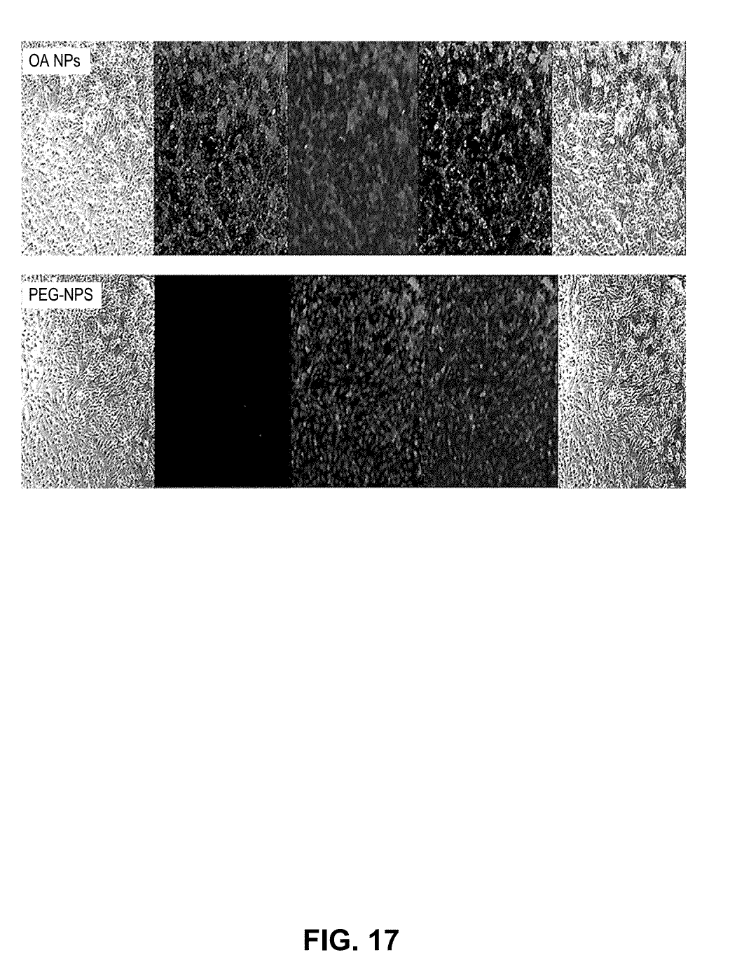

The uptake mechanism of FA-NPs was carried out using chemical inhibitors (FIG. 16) and immunofluorescent staining (FIG. 17). Based on the results of the chemical inhibitor study, CD36, caveolae, clathrin and scavenger receptor A appeared only weakly responsible in the uptake of OA-NPs into bEnd.3 cells. In contrast, scavenger receptor B-I is one of the main receptors responsible for the uptake of OA-NPs, as reflected by almost 50% in the reduction of cellular uptake after the use of BLT-1 and BLT-4. However, these endocytosis pathway are fairly general in nature and may not contribute to the biodistribution profile of OA-NPs in rat. Since no FATP chemical inhibitor has been reported, we utilized a FATP-4 antibody to determine whether FAT-4 participates in the uptake of OA-NPs in cells in vitro.

oleic acid is a substrate for FATP-4 and FATP-4 is highly expressed in the brain. bEnd.3 cells express FATP-4 but not FATP-1, as verified by reverse transcription-PCR (data not shown) and is consistent with the literature. The results of the fluorescent staining experiment described above, indicate co-localization of FATP-4 and OA-NPs. This indirect evidence supports the conclusion that FATP-4 participates in the uptake of OA-NPs into cells.

### Example 6

### Pharmacokinetic Profile of Coumarin 6-Loaded Nanoparticles in Plasma and Organs

Pharmacokinetic parameters of organs were calculated using WinNonlin 7.0.0.2535 (Pharsight Corp., CA, USA) with a non-compartmental model. For the pharmacokinetic parameters of plasma, a 2-compartmental model was used.

Male Sprague-Dawley® rats with a weight between 220 to 250 g were supplied by the Laboratory Animal Services Centre, The Chinese University of Hong Kong. The rats were housed at a 12/12 hour light/dark cycle with free access to water and standard laboratory chow. Coumarin 6-loaded NPs (30 mg/mL polymer concentration and 171 μM of coumarin 6) and coumarin 6 in 1% DMSO were injected into the rats through the left lateral tail vein (bolus injection, 136 μg/kg). After a designated time point, the rats were sacrificed by exsanguination via cardiac puncture after CO2 anesthesia. Blood was then collected, heparinized and centrifuged at 1000 G for 5 min for plasma collection. Organs including brain, liver, lung, kidneys, heart and spleen were collected after perfusion with cold 0.9% saline through the left ventricle (approx. 500 mL per rat). Organs were homogenized in saline. Coumarin 6 was extracted from the organ homogenate and plasma samples with acetonitrile. The concentrations of coumarin 6 in the extracts were quantified using a fluorescent microplate reader (Clariostar®, BMG Labtech, Germany) (Ex470-15 nm, Em507-15 nm, dichroic filter 485 nm). By spiking coumarin 6 standard solutions into blank organ homogenates/plasma before extraction, the percentage of recovery was determined to be >95%.

### Pharmacokinetics and Biodistribution

After identifying nanoparticles with potential for improved drug delivery into the brain (e.g., OA-NPs and LA-NPs) based on the preliminary screen results discussed herein, we investigated the corresponding plasma pharmacokinetics and biodistribution.

Male, SD-rats with a weight between 220 g to 250 g were used in this study. After intravenous injection of the OA-NP and LA-NP coumarin-6 formulations at 136 μg/kg, rats were scarified at 5 minutes, 30 minutes, 1 hour, 2 hours, 4 hours and 24 hours. Blood, brain, heart, liver, lung, kidneys and spleen were collected for analysis of coumarin-6 concentration.

### Plasma Pharmacokinetics

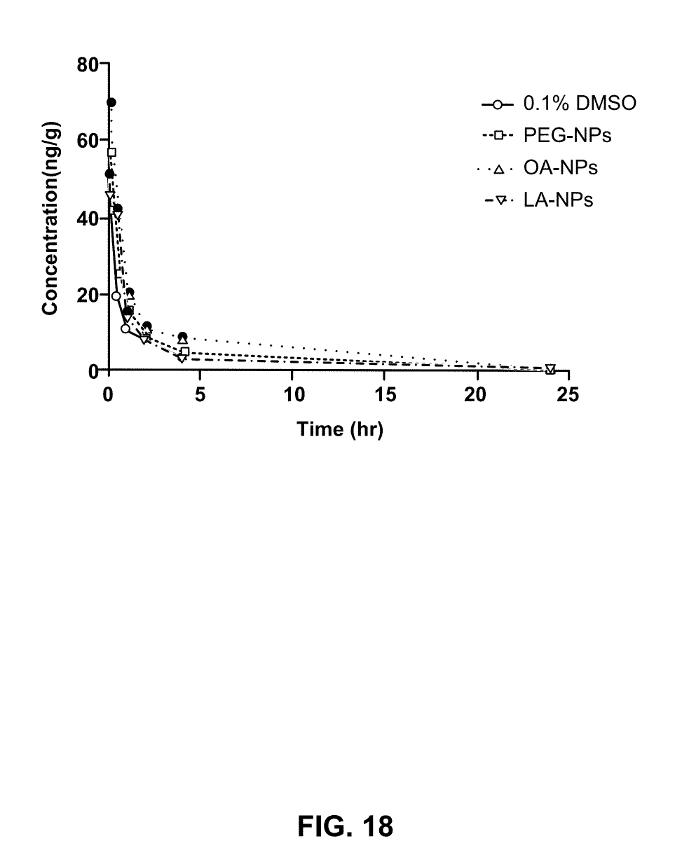

A two-compartment model was used to analyse the plasma pharmacokinetics of different coumarin-6 formulations in vivo (see, FIG. 18 and Table 1). Data were reported as mean±SEM. One-way ANOVA followed by Dunnett's multiple comparison test (α=0.05) against 0.1% DMSO. Statistical significant was defined as: p>0.05=not significance (ns), p≤0.05=*, p≤0.01=**, p≤0.001=***, p≤0.0001=****.

TABLE 1
Plasma pharmacokinetic profile of various FA-NPs

| | | | | | | CL | |
|---|---|---|---|---|---|---|---|
| | t_{1/2α} | t_{1/2β} | AUC_{0→∞} | V₁ | Vₛₛ | ((μg)/(ng/ | MRT |
| | (hr) | (hr) | (ng/ml * h) | (L) | (L) | ml)/h) | (hr) |
| 0.1% | 0.26 ± 0.08 | 2.28 ± 0.15 | 54.97 ± 5.08 | 0.64 ± 0.17 | 1.46 ± 0.20 | 0.56 ± 0.05 | 2.60 ± 0.15 |
| DMSO | | | | | | | |
| PEG- | 0.20 ± 0.05 | 1.97 ± 0.08 | 74.60 ± 7.81 | 0.42 ± 0.05 | 0.96 ± 0.07 | 0.42 ± 0.05 | 2.32 ± 0.09 |
| NPs | | | | | | | |
| OA-NPs | 0.46 ± 0.07* | 7.65 ± 0.26** | 174.16 ± 28.94* | 0.18 ± 0.03 | 1.56 ± 0.21 | 0.18 ± 0.03 | 8.67 ± 0.44 |
| LA-NPs | 0.65 ± 0.02*** | 47.18 ± 12.80*** | 148.48 ± 38.12 | 0.23 ± 0.06 | 8.36 ± 1.24*** | 0.23 ± 0.06 | 44.03 ± 15.60 |

The pharmacokinetic variables for intravenous injection of different coumarin-6 loaded FA-NP formulations were characterized by a short distribution half-life (t_{1/2α}), followed by a long distribution half-life (t_{1/2β}). OA-NPs and LA-NPs demonstrated longer t_{1/2α} and longer t_{1/2β} than PEG-NPs and 0.1% DMSO formulations. A longer absorption and distribution half-life lead to a 2-fold increase in AUC_{0→∞} indicating that OA-NPs and LA-NPs were retained better in the blood and distributed to the organs or surrounding tissue slower. This is a surprising result in view of current data in the literature. Coating nanoparticles with PEG general reduces reticuloendothelial system (RES) clearance and increases circulation time, compared to free drug molecules and nanoparticles without PEG. One explanation is that although the introduction of hydrophobic ligands increases hepatic clearances due to non-specific absorption of plasma protein. There are also examples in the literature where the presence of albumin on the surface (the major component of plasma protein) significantly improved the half-life of nanoparticles in the blood, by pH-dependent FcRn-mediated recycling pathway and large molecular size. Fatty acid, naturally present in the blood greatly reduces the risk of immunogenicity and toxicity compared to other ligands, can be used as an albumin-binding tag. Thus, the increase in blood half-life is likely to be contributed from protein binding on the surface of the nanoparticles.

### Brain Accumulation

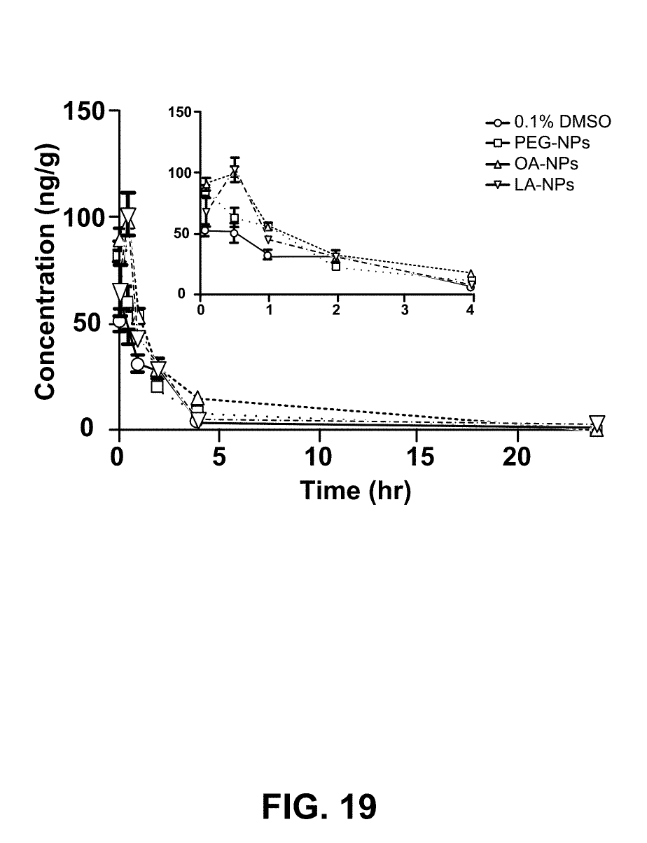

A non-compartment model was used to analysis the brain accumulation of different coumarin-6 formulation in vivo (see, FIG. 19 and Table 2). Data were reported as mean±SEM. One-way ANOVA followed by Dunnett's multiple comparison test (α=0.05) against 0.1% DMSO. Statistical significant was defined as: p>0.05 =not significance (ns), p≤0.05 =*, p≤0.01=**, p≤0.001=***, p≤0.0001=****.

TABLE 2
Brain pharmacokinetic profile of various FA-NPs

| | | | Cₘₐₓ | AUC_{0→∞} | MRT | Vz | CL |
|---|---|---|---|---|---|---|---|
| | t_{1/2}(hr) | tₘₐₓ(hr) | (ng/g) | (ng/g * h) | (hr) | (mg) | (mg/h) |
| 0.1% | 3.11 ± 0.27 | 0.22 ± 0.14 | 54.09 ± 6.84 | 154.70 ± 38.26 | 2.35 ± 0.43 | 0.96 ± 0.18 | 0.22 ± 0.06 |
| DMSO | | | | | | | |
| PEG- | 2.53 ± 0.05 | 0.083 ± 0 | 80.64 ± 6.30 | 192.18 ± 34.32 | 2.12 ± 0.26 | 0.61 ± 0.11 | 0.17 ± 0.04 |
| NPs | | | | | | | |
| OA-NPs | 2.41 ± 0.05 | 0.5 ± 0 | 97.88 ± 6.14* | 324.64 ± 27.88* | 2.59 ± 0.03 | 0.33 ± 0.04* | 0.09 ± 0.01* |
| LA-NPs | 5.94 ± 0.44*** | 0.5 ± 0 | 101.51 ± 10.11* | 252.62 ± 29.18 | 7.39 ± 0.98* | 1.03 ± 0.05** | 0.12 ± 0.02 |

For coumarin-6 in 0.1% DMSO and PEG-NPs, a fast distribution from the blood to the brain was observed as reflected from the short tₘₐₓ (0.08 to 0.22 hr), with negligible difference in the Cₘₐₓ and AUG_{0→∞}For LA-NPs and OA-NPs, a delayed tₘₐₓ (0.5 hr) was observed, together with a higher Cₘₐₓ than 0.1% DMSO and PEG-NPs. OA-NPs demonstrated a significant improved brain accumulation of coumarin-6 among all formulations, which was 2.1-fold better than 0.1% DMSO and 1.7-fold better than PEG-NP, based on the AUC_{0→∞}. Accordingly, OA-NPs can be used for drug delivery into the brain.

One interesting phenomenon observed was that LA-NPs showed significantly longer t_{1/2} and MRT than other formulations, together with larger Vz and lower CL. After further investigation, we concluded LA-NPs have a biphasic elimination profile versus a monophasic elimination profile observed in 0.1% DMSO, PEG-NPs and OA-NPs. The reason for a different elimination profile is unclear but could be useful formulation for the development of sustained drug delivery into the brain.

### Organ Distribution

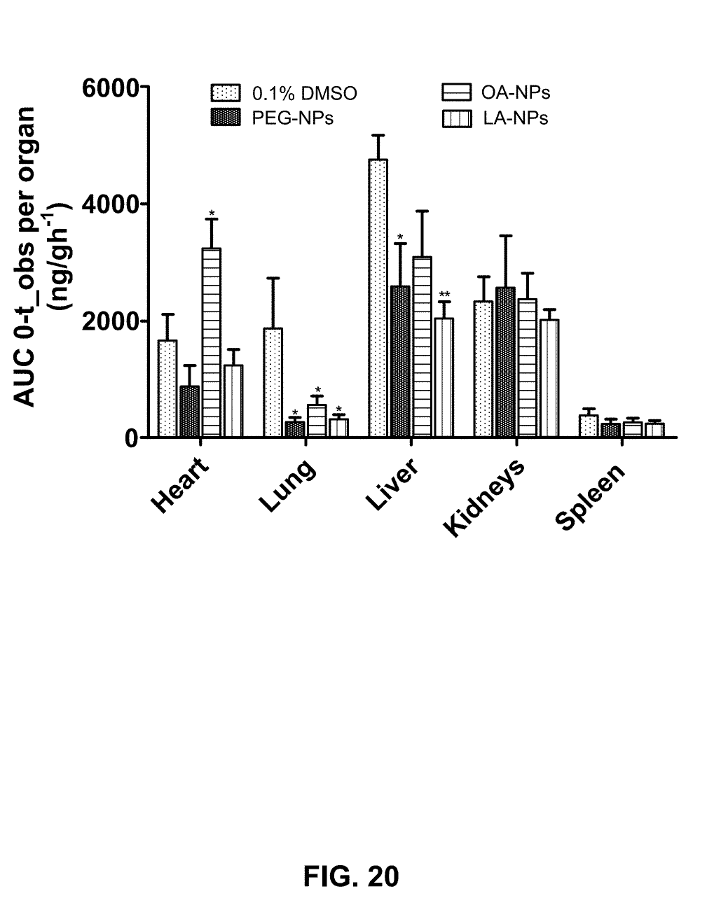

Accumulation of the various coumarin-6 formulations in other organs, including heart, lung, liver, kidneys and spleen were estimated based on the AUC 0-24 h_obs using a non-compartmental model (FIG. 20). The data was calculated based on the AUC 0-24 h_obs using a non-compartmental model. Data is reported as mean±SEM. One-way ANOVA followed by Dunnett's multiple comparison test (α=0.05) against 0.1% DMSO. Statistical significant was defined as: p>0.05=not significance (ns), p≤0.05=*, p≤0.01=**, p≤0.001 =***, p≤0.0001=****.

In general, the FA-NPs demonstrated a lower accumulation in the liver and lungs. The substantial decrease in lung accumulation is likely to be contributed to the presence of the pulmonary epithelial barrier and the presence of tight junctions dramatically reducing the uptake of FA-NPs into lung (see, Brune et al., Am. J. Physiol. Lung Cell Mol. Physiol., (2015), 308(8):L731-745). The reduction of liver accumulation is likely to be contributed to reduced uptake from the reticuloendothelial system (RES). Here, we observed PEG-NPs and LA-NPs showing a significant (statistically significant) reduction in liver accumulation as compared to 0.1% DMSO. OA-NPs showed a slightly non-significant difference; this could be due to the presence of FATP-4 in the liver tissue, which could counter the effect of reduced RES uptake. The improved accumulation in heart for OA-NPs over 0.1% DMSO is likely to be contributed to the presence of FATP-4 in heart tissue as well, while PEG-NPs and LA-NPs showed non-significant differences in heart accumulation as compared to 0.1% DMSO. No significant differences in the spleen and kidneys accumulation was observed for all FA-NPs.

### EXAMPLE 7

### Intracranial Distribution of Coumarin 6-Loaded Nanoparticles

After the brain was harvested according to the above procedure, it was placed on its dorsal surface in a Alto brain matrix (AL-1130, small rat coronal (175-300 g), Cellpoint Scientific Inc., Gaithersburg, MD, USA). Razor blades were carefully inserted through the cutting channels slicing the brain at right angles to the sagittal axis. The first blade was inserted at 5 mm and 14 mm of bregma and interaural, respectively. Next, two blades were inserted posterior to the first blade, along the caudal extent of the brain, at intervals of 3 mm. The forth blade was inserted posterior to the third blade at 4 mm interval. The last two blades were inserted posterior to the most caudal blade at intervals of 3 mm. The whole brain was thus divided into six sections. The blades were removed from the block carefully with coronal brain slices adhering to their surfaces. Seven brain regions, namely olfactory bulb (OB), cerecortex (COR), striatum (STR), hippocampus (HIP), thalamus and midbrain (THA), cerebellum (CERE) and brain stem (STEM), were then dissected from these slices using fine forceps and scalpels. Tissue was taken bilaterally for all brain regions.

The COR was a large cortical portion existing from section 2 to section 6. The OB consisted of the whole first section and lower part of the second section underneath the COR. The STR had very unique appearance with tiny white spots (white matter) on it and located at the lower part of section 3 and the middle part in section 4 surrounded by THA. The HIP was a narrow belt embedded between the THA and COR in section 4 and 5. THA were the remaining parts of section 4 and 5 containing thalamus, hypothalamus, midbrain and inferior colliculus in section 6. The CERE and STEM can be separated from each other in last section. After the brain was separated into 7 regions, the coumarin 6 content was quantified.

### Intracranial Distribution

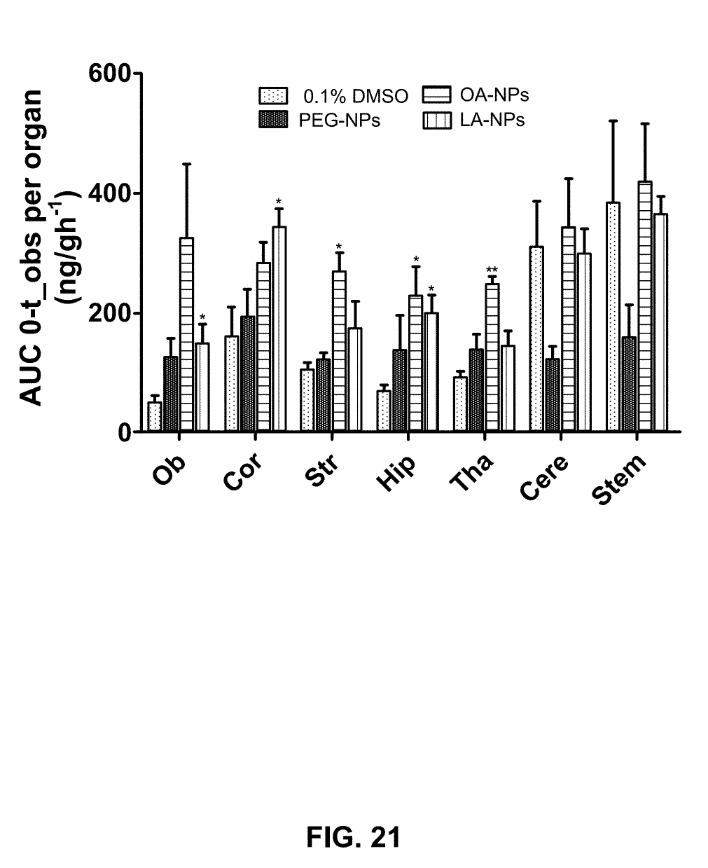

The accumulation of various coumarin-6 loaded FA-NP formulations in different regions of the brain, namely, olfactory bulb (OB), cerecortex (COR), striatum (STR), hippocampus (HIP), thalamus and midbrain (THA), cerebellum (CERE) and brain stem (STEM) were estimated based on the AUC 0-24 h_obs using a non-compartmental model (FIG. 21). The data was calculated based on the AUC 0-24 h_obs using a non-compartmental model. Data is reported as mean±SEM. One-way ANOVA followed by Dunnett's multiple comparison test (α=0.05) against 0.1% DMSO. Statistical significant was defined as: p>0.05=not significance (ns), p≤0.05 =*, p≤0.01=**, p≤0.001=***, p≤0.0001=****.

All four FA-NP formulations demonstrated similar brain intracranial distribution patterns and were evenly distributed with the brain, as demonstrated from the One-way ANOVA testing: 0.1% DMSO (F(6,7)=4.195,p=0.0411); PEG-NPs (F(6,7)=0.4179,p=0.8462); OA-NPs (F(6,7)=0.8169, p=0.5894) and LA-NPs ((F(6,7)=7.214, p=0.0099). Although both 0.1% DMSO and LA-NPs demonstrated statistically significant difference between groups (different brain sections) in One-way ANOVA, post-hoc test indicated there is no significant differences between groups.

Although the four FA-NP formulations were evenly distributed inside the brain, OA-NPs preferentially accumulated inside STR, HIP and THA, while LA-NPs preferentially accumulated inside OB, COR and HIP, as compared to 0.1% DMSO. In general, improvement of accumulation of OA-NPs and LA-NPs over 0.1% DMSO is observed in OB, STR, HIP, COR and THA, where enriched grey matter is present. We also noted that grey matter had a higher expression of FATP-1 (2-fold) and FATP-4 (8-fold) than in the white matter (data not shown).

All patents, patent applications, and other publications,.

All documents (for example, patents, patent applications, books, journal articles, or other publications) including GenBank Accession Numbers, cited in this application are incorporated by reference in the entirety for all purposes, to the same extent as if each individual document was specifically and individually indicated to be incorporated by reference in its entirety for all purposes. To the extent such documents incorporated by reference contradict the disclosure contained in the specification, the specification is intended to supersede and/or take precedence over any contradictory material.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

### REFERENCES

[1] M. Jove, M. Portero-Otin, A. Naudi, I. Ferrer, R. Pamplona, Metabolomics of human brain aging and age-related neurodegenerative diseases, J Neuropathol Exp Neurol 73(7) (2014) 640-57.
[2] R. W. Mitchell, N. H. On, M. R. Del Bigio, D. W. Miller, G. M. Hatch, Fatty acid transport protein expression in human brain and potential role in fatty acid transport across human brain microvessel endothelial cells, J Neurochem 117(4) (2011) 735-46.
[3] M. L. Adams, A. Lavasanifar, G. S. Kwon, Amphiphilic block copolymers for drug delivery, J Pharm Sci 92(7) (2003) 1343-55.
[4] G. S. Kwon, T. Okano, Soluble self-assembled block copolymers for drug delivery, Pharm Res 16(5) (1999) 597-600.
[5] W. H. De Jong, P. J. Borm, Drug delivery and nanoparticles:applications and hazards, Int J Nanomedicine 3(2) (2008) 133-49.
[6] B. D. Chithrani, A. A. Ghazani, W. C. Chan, Determining the size and shape dependence of gold nanoparticle uptake into mammalian cells, Nano Lett 6(4) (2006) 662-8.
[7] A. Noorafshan, S. Ashkani-Esfahani, A review of therapeutic effects of curcumin, Curr Pharm Des 19(11) (2013) 2032-46.
[8] S. Ji, Z. Zhu, T. R. Hoye, C. W. Macosko, Maleimide Functionalized Poly(epsilon-caprolactone)-b-poly(ethylene glycol) (PCL-PEG-MAL): Synthesis, Nanoparticle Formation, and Thiol Conjugation, Macromol Chem Phys 210(10) (2009) 823.
[9] H. Y. Li, Z. Chen, L. W. Ho, Q. Q. Li, P. S. Chan, C. H. Choi, W. Y. Lee, Oligonucleotides-conjugated Poly(ethylene oxide)-block-poly(c-caprolactone) (PEG-b-PCL) Nanoparticles as Drug Carriers Targeting Scavenger Receptor Class A, American Association of Pharmaceutical Scientist Annual Meeting and Exposition, Colorado, USA, 2016. Accepted.
[10] G. Basu Ray, I. Chakraborty, S.P. Moulik, Pyrene absorption can be a convenient method for probing critical micellar concentration (cmc) and indexing micellar polarity, J Colloid Interface Sci 294(1) (2006) 248-54.
[11] Q. Li, K. L. Lai, P. S. Chan, S.C. Leung, H. Y. Li, Y Fang, K. K. To, C.H. Choi, Q. Y. Gao, T. W. Lee, Micellar delivery of dasatinib for the inhibition of pathologic cellular processes of the retinal pigment epithelium, Colloids Surf B Biointerfaces 140 (2016) 278-86.
[12] J. Lu, S. C. Owen, M. S. Shoichet, Stability of Self-Assembled Polymeric Micelles in Serum, Macromolecules 44(15) (2011) 6002-6008.
[13] S. Yefimova, G. Gural'chuk, A. Sorokin, Y. Malyukin, I. Borovoy, A. Lubyanaya, Hydrophobicity effect on interactions between organic molecules in nanocages of surfactant micelle, J Appl Spectrosc 75(5) (2008) 658-663.
[14] F. Alexis, E. Pridgen, L. K. Molnar, O. C. Farokhzad, Factors affecting the clearance and biodistribution of polymeric nanoparticles, Mol Pharm 5(4) (2008) 505-15.
[15] M. Ukawala, T. Rajyaguru, K. Chaudhari, A. S. Manjappa, S. Pimple, A. K. Babbar, R. Mathur, A. K. Mishra, R. S. Murthy, Investigation on design of stable etoposide-loaded PEG-PCL micelles: effect of molecular weight of PEG-PCL diblock copolymer on the in vitro and in vivo performance of micelles, Drug Deliv 19(3) (2012) 155-67.
[16] H. Sun, R. Yang, J. Wang, X. Yang, J. Tu, L. Xie, C. Li, Q. Lao, C. Sun, Component-based biocompatibility and safety evaluation of polysorbate 80, RSC Advances 7(25) (2017) 15127-15138.
[17] R. D. Groot, K. L. Rabone, Mesoscopic simulation of cell membrane damage, morphology change and rupture by nonionic surfactants, Biophys J 81(2) (2001) 725-36.

1. A nanoparticle comprising a fatty acid conjugated to the surface of the nanoparticle and containing within the nanoparticle a therapeutic agent.

2. The nanoparticle of claim 1, wherein the fatty acid is selected from the group consisting of lauric acid (C12), myristic acid (C14), palmitic acid (C16), steric acid (C18), alpha-linolenic acid (ALA), linoleic acid (LA), oleic acid (OA), docosahexaenoic acid (DHA), erucic acid (EA), formic acid, acetic acid, propionic acid, butyric acid, isobutyric acid, valeric acid, isovaleric acid, and derivatives that contain one long alkyl chain in which the number of carbon varies from 2 to 5, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, stearidonic acid, arachidonic acid and derivatives that contain one long alkyl chain in which the number of carbon varies from 6 to 12, oxalic acid, malonic acid, succinic acid, glutaric acid, adipic acid, pimelic acid, suberic acid, azelaic acid, sebacic acid and derivatives that contain one long alkyl chain in which the number of carbon varies from 8 to 20, citric acid, and tricarboxylic acid and its derivatives, omega-3, fatty acids, wherein the omega-3 fatty is hexadecatrienoic acid,a-Linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, and tetracosahexaenoic acid, omega-6 fatty acids, wherein the omega-6 fatty acid is linoleic acid, gamma-linolenic acid, calendic acid, eicosadienoic acid, dihomoamma-linolenic acid, arachidonic acid, docosadienoic acid, adrenic acid, osbond acid, tetracosatetraenoic acid, and tetracosapentaenoic acid, omega-9 fatty acids, wherein the omega-9 fatty acid is oleic acid, elaidic acid, gondoic acid, mead acid, erucic acid, nervonic acid, and ximenic acid.

3.-4. (canceled)

5. The nanoparticle of claim 1, wherein the nanoparticle is a polymeric nanoparticle comprising a plurality of polymer blocks, wherein the plurality of polymer blocks comprises one or more polymers selected from the group consisting of polylactic acid (PLA), polyethylene glycol (PEG) or polyethylene oxide (PEO), polycaprolactone (PCL), methoxypolyethylene glycol (MPEG), Poly D, L-glycolide (PLG), polycyanoacrylate (PCA), polylactic-co-glycolic acid (PLGA), polyvinyl alcohol (PVA), polyvinylpyrrolidone, polybutadiene (PBD), methyl methacrylate(MMA), methacrylic acid (MAA), d-α-tocopheryl polyethylene glycol 1000 succinate, PEG-PLA, PEG-PLLLA, PEG-PDLLA, PEG-PDDLA, mPEG-PLA, mPEG-PLLLA, mPEG-PDLLA, mPEG-PDDLA, PEG-PCL, PEG-PLGA, PEG-PCL, mPEG-PCL, PEG-DPSE, mPEG-DPSE, PEO-PBD, mPEO-PBD, Pluronics (PEO-PPO-PEO), PLGA-PEG-PLGA, PEG-PLGA-PEG, PEG-PCL-PEG, PCL-PEG-PCL, Vitamin E-TPGS, Solutol HS15, and Soluplus, or a combination thereof.

6. The nanoparticle of claim 5 wherein the polymeric nanoparticle comprises a diblock copolymer, wherein said diblock copolymer comprises (i) a first block of hydrophobic polymer and (ii) a second block of hydrophilic polymer.

7.-8. (canceled)

9. The nanoparticle of claim 5 wherein the polymeric nanoparticle is a poly(ethylene glycol)-block-poly(epsilon-caprolactone) (PEG-b-PCL) nanoparticle.

10. The nanoparticle of claim 1, wherein the nanoparticle is selected from the group consisting of a liposome, solid lipid nanoparticle, gold nanoparticle, silver nanoparticle, iron nanoparticle, Gd nanoparticle, polystyrene nanoparticle, albumin nanoparticle, chitosan and derivative nanoparticles, and a dendrimer.

11.-12. (canceled)

13. The nanoparticle of claim 1, wherein the nanoparticle has an average mean particle size of about 25 nm to about 125 nm.

14. (canceled)

15. The nanoparticle of claim 1, wherein the nanoparticle targets delivery of the therapeutic agent across the blood brain barrier.

16. The nanoparticle of claim 1, wherein the therapeutic agent is a chemotherapeutic agent, antibiotic, antiviral drug, vaccine, diagnostic agent, monoclonal antibody or a binding fragment thereof, neuropeptide, CNS stimulant, anticonvulsant, antiemetic/anti-vertigo agent, muscle relaxant, narcotic analgesic, nonnarcotic analgesic, sedative, anti-inflammatory agents, cholinergic agonist, cholinesterase inhibitor, general anesthetic, imaging agent, or drug for the prevention, treatment, and diagnosis of Parkinson's disease, Alzheimer's disease, dementia, stroke, brain cancer, inflammatory and infectious diseases of the central nervous systems.

17. The nanoparticle of claim 16, wherein the therapeutic agent is selected from the group consisting of aldesleukin (proleukin), altretamine (hexalen), amsacrine, ara-c cytarabine: cytarabine (ara-c), anastrazole, asparaginase, azacytidine, azidothymidine, carmustine, bendamustine, bevacizumab, bromocriptine, buserelin, busulfan, cabergolin, calcium folinate (leucovorin), camptosar (irinotecan), camptosar (irinotecan), capecitabine (xeloda), carboplatin (paraplatin), ccnu (lomustine), chloramucil (leukeran), cisplatin, cladribine (leustatin), clofarabine, cytosine arabinoside, cytarabine, cytoxin (cyclophosphamide), dacarbazine, dactinomycin, daunorubicin, decitibine, dexrazoxan, docetaxel (taxotere), doxorubicin hydrochloride (hydroxydaunorubicin), epirubicin, erlotinib (tarceva), estramustine, etoposide, exemestane (aromasin), fludarabine, fluorodeoxyuridine, 5-fluorouracil, flutamide, fulvestrant, gemcitabine, goserelin (zoladex), herceptin, hydroxyurea, idarubicin, ifosfamide, imatinib, interferon, ixempra (ixabepilone), lanvis thioguanine, lapatinib ditosylate (tykerb), lenalidomide (revlimid), letrozole (femara), luprone (luprolide), lomustine, lysodren, mechlorethamine hydrochloride, mitotan, megastrol, melphalan, mesna uromitexan, mercaptopurine, methotrexate, mitomycin, mitoxantrone, mitotane, navelbine vinerelobine, nelarabine, novladex, omustine, oxaliplatin, paclitaxel, panitumumab, paraplatin (carboplatin), patipilone epithilone b, pharmorubicin epirubicin, photofrin porfimer, pentostatin, procarbazine hydrochloride (natulan), trans-retinoic acid, rituxan (rituximab), somatuline lanreotide, streptozocin, sunitinib malate (sutent), tamoxifen, temodal temozolomide (temodar), teniposide, testosterone, topotecan, thioguanine, traztuzumab, thalidomide, thiotepa, tretinoin, vinblastine, vincristine, vepesid etoposide, vinorelbine, vindesine, and vorinostat.

18.-19. (canceled)

20. The nanoparticle of claim 1, wherein the nanoparticle has a polydispersity index (PDI) of less than 0.5.

21.-24. (canceled)

25. The nanoparticle of claim 1, wherein the nanoparticle improves solubility of the therapeutic agent across the blood brain barrier.

26. The nanoparticle of claim 1, wherein the nanoparticle is a micelle nanoparticle.

27.-29. (canceled)

30. The nanoparticle of claim 1, wherein the therapeutic agent is present at a drug loading of between 1% and 99.99%.

31. The nanoparticle of claim 1, wherein the nanoparticle comprises an encapsulation efficiency for the therapeutic agent of between 1% to 99%.

32. A method for delivering a therapeutic agent by administering to a subject in need thereof the nanoparticle of claim 1.

33. The method of claim 32, wherein the nanoparticle is a polymeric nanoparticle.

34.-35. (canceled)

36. The method of claim 33, wherein the polymeric nanoparticle is a poly(ethylene glycol)-block-poly(epsilon-caprolactone) (PEG-b-PCL) nanoparticle.

37.-39. (canceled)

40. The method of claim 32, wherein the therapeutic agent is used to treat Alzheimer's disease, Parkinson's disease, Huntingdon's disease, schizophrenia, epilepsy, stroke, traumatic brain injury, encephalitis, meningitis, depression, neuroblastomas, multiple sclerosis (MS), prion disease, amyotrophic lateral sclerosis (ALS), transverse myelitis, motor neuron disease, Pick's disease, Lyme disease, brain tumors, and spinal cord tumors.

41.-42. (canceled)

43. The method of claim 32, wherein the administering comprises parenteral, intramuscular, subcutaneously, intranasal, intrathecal, intraparenchymal, intracerebroventricular, peroral, intracranial administration, and intraperitoneal administration.
